SEQUENCE LISTING (68 sequences)

SEQ ID NO: 1 - protein, 5 aa, Artificial Sequence
  Pitrilysin consensus sequence
HXXEH

SEQ ID NO: 2 - protein, 46 aa, Artificial Sequence
  Pitrilysin consensus sequence
GXXHXXEHXXXXGXXXXXXXXXXXXXXXXXXXXXXXXNAXTXXXXT

SEQ ID NO: 3 - protein, 46 aa, Artificial Sequence
  Pitrilysin consensus sequence
GXXHXXEHXXXXGXXKYPXXXXXXXXXXLXXXXXXXXNAXTXXXXT

SEQ ID NO: 4 - protein, 989 aa, Saccharomyces cerevisiae
MLRFQRFASSYAQAQAVRKYPVGGIFHGYEVRRILPVPELRLTAVDLVHSQTGAEHLHIDRDDKNNVFSIAFKTNPPDSTGVPHILEHTTLCGSVKYPVRDPFFKMLNKSLANFMNAMTGPDYTFFPFSTTNPQDFANLRGVYLDSTLNPLLKQEDFDQEGWRLEHKNITDPESNIVFKGVVYNEMKGQISNANYYFWSKFQQSIYPSLNNSGGDPMKITDLRYGDLLDFHHKNYHPSNAKTFTYGNLPLVDTLKQLNEQFSGYGKRARKDKLLMPIDLKKDIDVKLLGQIDTMLPPEKQTKASMTWICGAPQDTYDTFLLKVLGNLLMDGHSSVMYQKLIESGIGLEFSVNSGVEPTTAVNLLTVGIQGVSDIEIFKDTVNNIFQNLLETEHPFDRKRIDAIIEQLELSKKDQKADFGLQLLYSILPGWTNKIDPFESLLFEDVLQRFRGDLETKGDTLFQDLIRKYIVHKPCFTFSIQGSEEFSKSLDDEEQTRLREKITALDEQDKKNIFKRGILLQEKQNEKEDLSCLPTLQIKDIPRAGDKYSIEQKNNTMSRITDTNGITYVRGKRLLNDIIPFELFPYLPLFAESLTNLGTTTESFSEIEDQIKLHTGGISTHVEVTSDPNTTEPRLIFGFDGWSLNSKTDHIFEFWSKILLETDFHKNSDKLKVLIRLLASSNTSSVADAGHAFARGYSAAHYRSSGAINETLNGIEQLQFINRLHSLLDNEETFQREVVDKLTELQKYIVDTNNMNFFITSDSDVQAKTVESQISKFMERLPHGSCLPNGPKTSDYPLIGSKCKHTLIKFPFQVHYTSQALLGVPYTHKDGSALQVMSNMLTFKHLHREVREKGGAYGGGASYSALAGIFSFYSYRDPQPLKSLETFKNSGRYILNDAKWGVTDLDEAKLTIFQQVDAPKSPKGEGVTYFMSGVTDDMKQARREQLLDVSLLDVHRVAEKYLLNKEGVSTVIGPGIEGKTVSPNWEVKEL

SEQ ID NO: 5 - protein, 882 aa, Schizosaccharomyces pombe
MNYAKLSIAFSKKTIKTHNCRLFQRWLHVGDKVHDFRVVDTKKVPELQLNYTRLKHEPTNADMIHLDREDPNSVFSIGFQTPAENDEGIPHILEHTTLCGSNKYPVRDPFFKMLNRSLATFMNAFTASDFTFYPFATVNTTDYKNLRDVYLDATLFPKLRKLDFLQEGWRFEHADVNDKKSPIIFNGVVYNEMKGQVSDSSYIFYMLFQQHLFQGTAYGFNSGGDPLAIPDLKYEELVKFHRSHYHPSNAKILSYGSFPLEDNLSALSETFRPFSKRELNLPNTFLKEFDQEKRVVEYGPLDPVMAPGRQVKTSISFLANDTSNVYETFALKVLSKLCFDGFSSPFYKALIESGLGTDFAPNSGYDSTTKRGIFSVGLEGASEESLAKIENLVYSIFNDLALKGFENEKLEAILHQMEISLKHKSAHFGIGLAQSLPFNWFNGADPADWLSFNKQIEWLKQKNSDGKLFQKLIKKYILENKSRFVFTMLPSSTFPQRLQEAEAKKLQERTSKLTDEDIAEIEKTSVKLLEAQSTPADTSCLPTLSVSDIPETIDETKLKFLDIAGMKAQWYDLAAGLTYIRLLLPLKNFPESLIPYLPVYCDACLNLGTHSESIGDLEHQIRRYTGGISISPSAVTNNSDVSKYELGIAISGYALDKNVGKLVELINKAFWNTNLSNTDKLAIMLKTSVSGITDGIAEKGHSFAKVSSASGLTEKTSITEQLGGLTQVKLLSQLSREESFGPLVEKLTAIREILRGTSGFKAAINASPTQHEVVEKALQKFMKSRGVNQQTQTKSTSKERNGINSIKTYHELPFQTYFAAKSCLGVPYTHPDGAPLQILSSLLTHKYLHGEIREKGGAYGAGLSYSGIDGVLSFFTYRDSDP

SEQ ID NO: 6 - protein, 973 aa, Clostridium perfringens
MNFKENNIYSGFKLLNIENLNEIGGVGLRFEHEKTKAKLIKILSEDDNKCFAIGFRTPPENSTGVPHILEHSVLCGSRKFNTKEPFVELLKGSLNTFLNAMTYPDKTIYPVASRNEKDFMNLMDVYLDAVLYPNIYKHKEIFMQEGWHYYIENKEDELKYNGVVYNEMKGAYSSPDSILYRKIPQTIYPDTCYALSSGGDPDEIPNLTYEEFVEFHKKYYHPSNSYIFLYGNGDTEKELEFINEEYLKNFEYKEIDSEIKEQKSFESMKEESFTYGIAESEDLNHKSYYSLNFVIGDATDGEKGLAFDVLAYLLTRSTAAPLKKALIDAGIGKAVSGDFDNSTKQSAFTVLVKNAELNKEEEFKKVVMDTLKDLVENGIDKELIEASINRVEFELREGDYGSYPNGLIYYLKVMDSWLYDGDPYVHLEYEKNLEKIKSALTSNYFEDLIERYMINNTHSSLVSLHPEKGINEKKSAELKKKLEEIKNSFDEKTLNEIIDNCKKLKERQSTPDKKEDLESIPMLSLEDIDKEATKIPTEEKEIDGITTLHHDFHTNKIDYVNFFFNTNSVPEDLIPYVGLLCDILGKCGTENYDYSKLSNAINISTGGISFGAITFANLKKNNEFRPYLEISYKALSSKTNKAIELVDEIVNHTDLDDMDRIMQIIREKRARLEGAIFDSGHRIAMKKVLSYSTNRGAYDEKISGLDYYDFLVNIEKEDKKSTISDSLKKVRDLIFNKGNMLISYSGKEEEYENFKEKVKYLISKTNNNDFEKEEYNFELGKKNEGLLTQGNVQYVAKGGNYKTHGYKYSGALSLLESILGFDYLWNAVRVKGGAYGVFSNFRRDGGAYIVSYRDPNIKSTLEAYDNIPKYLNDFEADEREMTKYIIGTIRKYDQPISNGIKGDIAVSYYLSNFTYEDLQKEREEIINADVEKIKSFAPMIKDLMKEDYICVLGNEEKIKENKDLFNNIKSVIK

SEQ ID NO: 7 - protein, 971 aa, Borrelia burgdorferi
MKKKKIFKLISKTYLEEHDAEGYYFKHESGLEVFHLKSDSFKENAFCIAFKTIPSNNTGVAHVLEHTIFCGSSKYKIKDPFLYLLKGSLNTFLNAMTFPDKTIYPAASTIEKDYFNLFNIYADSIFNPLLKKESFMQEGYNINPKDFKVSGIVFNEMKGSYSNKNSLINEIVSSSLFEEGAYKYDSGGIPTNIIDLTYESFLDFYKKYYTLENCKIFLCGNTQTEKNLNFIEKYIIRPYKKEKSNVNINIENVKRWEKGKKLTYKIPKENDNSLGVYTINWLCTEINNIEDSIGLEILSEILLDDSCSFTINILKSGIGEDIAHISGINTDLKESIFSFGLQNVVENKEKEFKNLVFSELKNLVKNKIPKELIKGILFGYEFALKEEKGQNFPIALMIKSFKGWLNGLHPIKTLQTSYYINEITNKLEKGIYYFENLIEKYLIFNNHYTLISFIPSHDTEKEMEEEIEKKLMAREIEIKQNPEEFLQFKKDYNQFKKYQNKKDSKADIAKLPLLKIEDLPKQIEKSLDLNEIKELNLHSFKFKSNNIFNVNLFFKLDFLEKEDYIYLSLFKRALQDLSTKNYSYININNKIQNTLGQINISESYDEDIDGNILNSFNISFKSFNNKVKESFELIKEILININFHDYERLKEITLSLKNDFKSLLIPKGHLLAMLRSKSKLKLNEYLKELQNGITGREFWQKAKTDTESLKEIANKLDNLKNKIILKNNLSALIMGNTDDILKNLENEFFNLKESLEESNHYNGLLNLDANSKALREIIIIQSKVAFNAICFPSYKINDENYPKANFLEHVLRSGIFWEKIRVMGGAYGASASIANGIFSFASYRDPNFTKTYQAFEKSLEELANNKMTDDEIYTYLIGLIGTNIYVKTKATEALQSYRRKMLNISDSLRQDIRNAYFTITPQDIKEISTKILTQIRQHNSIASLVNNQIYEEEKNNLEKLIGKEYSLKKIY

SEQ ID NO: 8 - protein, 995 aa, Caenorhabditis elegans
MSASKLWSCTETVLNGGIKLFLYSSKNTKLRVAIGEVPGPMVHGAVSFVTEADSDDGLPHTLEHLVFMGSKKYPFKGVLDVIANRCLADGTNAWTDTDHTAYTLSTVGSDGFLKVLPVYINHLLTPMLTASQFATEVHHITGEGNDAGVVYSEMQDHESEMESIMDRKTKEVIYPPFNPYAVDTGGRLKNLRESCTLEKVRDYHKKFYHLSNMVVTVCGMVDHDQVLEIMNNVENEHMSTVPDHFPKPFSFALSDIKESTVHRVECPTDDASRGAVEVAWFAHSPSDLETHSSLHVLFDYLSNTSVAPLQKDFILLEDPLASSVSFHIAEGVRCDLRLNFAGVPVEKLDECAPKFFDKTVREHLEEANFDMERMGYLIDQTILNELVKLETNAPKDIMSHIIGHQLFDNEDEELFKKRTNEIDFLKKLKSEPASYWVQLVNKYFTAPSATVIGVPNEELVDKIAEEEEKRIAAQCEKLGKKGLEEAGKSLEAAILENTANHPSAELLDQLIVKDLEAFDRFPVQSLTSNSPSLTPQQSTFLAQFPFHANLHNCPTKFVEIFFLLDSSNLSIEDRSYLFLYTDLLFESPAMIDGVLTSADDVAKHFTKDLIDHSIQVGVSGLYDRFVNLRIKVGADKYPLLAKWAQIFTQGVVFDPSRIHQCAQKLAGEARDRKRDGCTVASTAVASMVYGKNTNCILFDELVLEKLHEKISKDVMKNPEAVLEKLEQVRSALFSNGVNAHFVADVDSIDPKMLSSDLWTWVQADPRFGPGHQFSAEAGENVSLELGKELLIGVGGSESSFIYQTSFLDANWNSEELIPAMIFGQYLSQCEGPLWRAIRGDGLAYGANVFVKPDRKQITLSLYRCAQPAVAYERTRDIIRKIVESGEISKAEFEGAKRSTVFEMMKREGTVSGAAKISILNNFRQTPHPFNIDLCRRIWNLTSEEMVKIGGPPLARLFDEKCFVRSIAVHPSKLNEMKKAFPGSSKIKISDLQFAC

SEQ ID NO: 9 - protein, 962 aa, Escherichia coli
MPRSTWFKALLLLVALWAPLSQAETGWQPIQETIRKSDKDNRQYQAIRLDNGMVVLLVSDPQAVKSLSALVVPVGSLEDPEAYQGLAHYLEHMSLMGSKKYPQADSLAEYLKMHGGSHNASTAPYRTAFYLEVENDALPGAVDRLADAIAEPLLDKKYAERERNAVNAELTMARTRDGMRMAQVSAETINPAHPGSKFSGGNLETLSDKPGNPVQQALKDFHEKYYSANLMKAVIYSNKPLPELAKMAADTFGRVPNKESKKPEITVPVVTDAQKGIIIHYVPALPRKVLRVEFRIDNNSAKFRSKTDELITYLIGNRSPGTLSDWLQKQGLVEGISANSDPIVNGNSGVLAISASLTDKGLANRDQVVAAIFSYLNLLREKGIDKQYFDELANVLDIDFRYPSITRDMDYVEWLADTMIRVPVEHTLDAVNIADRYDAKAVKERLAMMTPQNARIWYISPKEPHNKTAYFVDAPYQVDKISAQTFADWQKKAADIALSLPELNPYIPDDFSLIKSEKKYDHPELIVDESNLRVVYAPSRYFASEPKADVSLILRNPKAMDSARNQVMFALNDYLAGLALDQLSNQASVGGISFSTNANNGLMVNANGYTQRLPQLFQALLEGYFSYTATEDQLEQAKSWYNQMMDSAEKGKAFEQAIMPAQMLSQVPYFSRDERRKILPSITLKEVLAYRDALKSGARPEFMVIGNMTEAQATTLARDVQKQLGADGSEWCRNKDVVVDKKQSVIFEKAGNSTDSALAAVFVPTGYDEYTSSAYSSLLGQIVQPWFYNQLRTEEQLGYAVFAFPMSVGRQWGMGFLLQSNDKQPSFLWERYKAFFPTAEAKLRAMKPDEFAQIQQAVITQMLQAPQTLGEEASKLSKDFDRGNMRFDSRDKIVAQIKLLTPQKLADFFHQAVVEPQGMAILSQISGSQNGKAEYVHPEGWKVWENVSALQQTMPLMSEKNE

SEQ ID NO: 10 - protein, 1161 aa, Homo sapiens
MLRRVAVAAVFATGRKLRCEAGRDVTAVGRIEARGLCEESAKPFPTLTMPGRNKAKSTCSCPDLQPNGQDLGESGRVARLGADESEEEGRSLSNVGDPEIIKSPSDPKQYRYIKLQNGLQALLISDLSNVEGKTGNATDEEEEEEEEEEEGEEEEEEEEDDDDDDDEDSGAEIQDDDEEGFDDEEEFDDDEHDDDDLDNEENELEELEERVEARKKTTEKQSAAALCVGVGSFADPDDLPGLAHFLEHMVFMGSLKYPDENGFDAFLKKHGGSDNASTDCERTVFQFDVQRKYFKEALDRWAQFFIHPLMIRDAIDREVEAVDSEYQLARPSDANRKEMLFGSLARPGHPMGKFFWGNAETLKHEPKKNNIDTHARLREFWMRYYSAHYMTLVVQSKETLDTLEKWVTEIFSQIPNNGLPKPNFSHLTDPFDTPAFNKLYRVVPIRKIHALTITWALPPQQQHYRVKPLHYISWLVGHEGKGSILSYLRKKCWALALFGGNGETGFEQNSTYSVFSISITLTDEGYEHFYEVAHTVFQYLKMLQKLGPEKRVFEEIQKIEDNEFHYQEQTDPVEYVENMCENMQLYPRQDFLTGDQLLFEYKPEVIAEALNQLVPQKANLVLLSGANEGRCDLKEKWFGTQYSIEDIENSWTELWKSNFDLNSDLHLPAENKYIATDFTLKAFDCPETEYPAKIVNTPQGCLWYKKDNKFKIPKAYIRFHLISPLIQKSAANVVLFDIFVNILTHNLAEPAYEADVAQLEYKLVAGEHGLIIRVKGFNHKLPLLFQLIIDYLTEFSSTPAVFTMITEQLKKTYFNILIKPETLAKDVRLLILEYSRWSMIDKYRALMDGLSLESLLNFVKDFKSQLFVEGLVQGNVTSTESMDFLRYVVDKLNFVPLEREMPVQFQVVELPSGHHLCKVRALNKGDANSEVTVYYQSGTRSLREYTLMELLVMHMEEPCFDFLRTKQTLGYHVYPTCRNTSGILGFSVTVGTQATKYNSETVDKKIEEFLSSFEEKIENLTEDAFNTQVTALIKLKECEDTHLGEEVDRNWNEVVTQQYLFDRLAHEIEALKSFSKSDLVSWFKAHRGPGSKMLSVHVVGYGKYELEEDGAPVCEDPNSREGMQLIYLPPSPLLAESTTPITDIRAFTATLSLFPYHKIVK

SEQ ID NO: 11 - protein, 1019 aa, Homo sapiens
MRYRLAWLLHPALPSTFRSVLGARLPPPERLCGFQKKTYSKMNNPAIKRIGNHITKSPEDKREYRGLELANGIKVLLMSDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQEWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTVMSKLWFKQDDKKKKPKACLNFEFFSPFAYVDPLHCNMAYLYLELLKDSLNEYAYAAELAGLSYDLQNTIYGMYLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQSLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVDAPRRHKVSVHVLAREMDSCPVVGEFPCQNDINLSQAPALPQPEVIQNMTEFKRGLPLFPLVKPHINFMAAKL

SEQ ID NO: 12 - protein, 1265 aa, Arabidopsis thaliana
MASSSSSIFTGVKFSPILAPFNSGDSRRSRYLKDSRNKVRFNPSSPRLTPHRVRVEAPSLIPYNGLWAAQPNSHKGRLKRNIVSGKEATGISLSQGRNFCLTCKRNQAGIRRALPSAFVDRTAFSLSRSSLTSSLRKHSQIVNATLGPDEPHAAGTAWPDGIVAERQDLDLLPPEIDSAELEAFLGCELPSHPKLHRGQLKNGLRYLILPNKVPPNRFEAHMEVHVGSIDEEEDEQGIAHMIEHVAFLGSKKREKLLGTGARSNAYTDFHHTVFHIHSPTHTKDSEDDLFPSVLDALNEIAFHPKFLSSRVEKERRAILSELQMMNTIEYRVDCQLLQHLHSENKLGRRFPIGLEEQIKKWDVDKIRKFHERWYFPANATLYIVGDIDNIPRIVHNIEAVFGKNGLDNESTPSSPSPGAFGAMANFLVPKLPAGLGGTFSNEKTNTADQSKMIKRERHAIRPPVEHNWSLPGTSVDLKPPQIFKHELLQNFAINMFCKIPVSKVQTFGDLRNVLMKRIFLSALHFRINTRYKSSNPPFTSVELDHSDSGREGCTVTTLTVTAEPQNWQNAVKVAVQEVRRLKEFGVTRGELTRYMDALLKDSEHLAAMIDNVSSVDNLDFIMESDALSHTVMDQTQGHETLVAVAGTVTLEEVNTVGAKVLEFISDFGRPTALLPAAIVACVPTKVHVDGVGESDFNISPDEIIESVKSGLLAPIEAEPELEVPKELISQSQLKELTLQRNPCFVPIPGSGLTKLHDKETGITQLRLSNGIAVNYKKSTTESRAGVMRLIVGGGRAAETSDSKGAVVVGVRTLSEGGRVGDFSREQVELFCVNHLINCSLESTEEFIAMEFRFTLRDNGMQAAFQLLHMVLERSVWLEDAFDRARQLYLSYFRSIPKSLERATAHKLMIAMLNGDERFVEPTPKSLQSLNLESVKDAVMSHFVGDNMEVSIVGDFSEEEIERCILDYLGTVKASHDSAKPPGSEPILFRQPTAGLQFQQVFLKDTDERACAYIAGPAPNRWGFTVDGDDLFQSVSKLPVAHDGLLKSEEQLLEGGDRELQKKLRAHPLFFGVTMGLLAEIINSRLFTTVRDSLGLTYDVSFELNLFDRLKLGWYVISVTSTPGKVYKAVDACKNVLRGLHSNQIAPRELDRAKRTLLMRHEAELKSNAYWLNLLAHLQASSVQRKELSCIKELVSLYEAASIEDIYLAYNQLRVDEDSLYSCIGIAGAQAGEEITVLSEEEEPEDVFSGVVPVGRGSSMTTRPTT

SEQ ID NO: 13 - protein, 534 aa, Homo sapiens
MRPDDKYHEKQAQVEATKLKQKVEALSPGDRQQIYEKGLELRSQQSKPQDASCLPALKVSDIEPTIPVTELDVVLTAGDIPVQYCAQPTNGMVYFRAFSSLNTLPEELRPYVPLFCSVLTKLGCGLLDYREQAQQIELKTGGMSASPHVLPDDSHMDTYEQGVLFSSLCLDRNLPDMMQLWSEIFNNPCFEEEEHFKVLVKMTAQELANGIPDSGHLYASIRAGRTLTPAGDLQETFSGMDQVRLMKRIAEMTDIKPILRKLPRIKKHLLNGDNMRCSVNATPQQMPQTEKAVEDFLRSIGRSKKERRPVRPHTVEKPVPSSSGGDAHVPHGSQVIRKLVMEPTFKPWQMKTHFLMPFPVNYVGECIRTVPYTDPDHASLKILARLMTAKFLHTEIREKGGAYGGGAKLSHNGIFTLYSYRDPNTIETLQSFGKAVDWAKSGKFTQQDIDEAKLSVFSTVDAPVAPSDKGMDHFLYGLSDEMKQAHREQLFAVSHDKLLAVSDRYLGTGKSTHGLAILGPENPKIAKDPSWIIR

SEQ ID NO: 14 - protein, 409 aa, Bacillus subtilis
MIKRYTCPNGVRIVLENNPTVRSVAIGVWIGTGSRHETPEINGISHFLEHMFFKGTSTKSAREIAESFDRIGGQVNAFTSKEYTCYYAKVLDEHANYALDVLADMFFHSTFDENELKKEKNVVYEEIKMYEDAPDDIVHDLLSKATYGNHSLGYPILGTEETLASFNGDSLRQYMHDYYTPDRVVISVAGNISDSFIKDVEKWFGSYEAKGKATGLEKPEFHTEKLTRKKETEQAHLCLGFKGLEVGHERIYDLIVLNNVLGGSMSSRLFQDVREDKGLAYSVYSYHSSYEDSGMLTIYGGTGANQLQQLSETIQETLATLKRDGITSKELENSKEQMKGSLMLSLESTNSKMSRNGKNELLLGKHKTLDEIINELNAVNLERVNGLARQLFTEDYALALISPSGNMPS

SEQ ID NO: 15 - protein, 438 aa, Mycobacterium tuberculosis
MPRRSPADPAAALAPRRTTLPGGLRVVTEFLPAVHSASVGVWVGVGSRDEGATVAGAAHFLEHLLFKSTPTRSAVDIAQAMDAVGGELNAFTAKEHTCYYAHVLGSDLPLAVDLVADVVLNGRCAADDVEVERDVVLEEIAMRDDDPEDALADMFLAALFGDHPVGRPVIGSAQSVSVMTRAQLQSFHLRRYTPERMVVAAAGNVDHDGLVALVREHFGSRLVRGRRPVAPRKGTGRVNGSPRLTLVSRDAEQTHVSLGIRTPGRGWEHRWALSVLHTALGGGLSSRLFQEVRETRGLAYSVYSALDLFADSGALSVYAACLPERFADVMRVTADVLESVARDGITEAECGIAKGSLRGGLVLGLEDSSSRMSRLGRSELNYGKHRSIEHTLRQIEQVTVEEVNAVARHLLSRRYGAAVLGPHGSKRSLPQQLRAMVG

SEQ ID NO: 16 - DNA, 34 bp, Artificial Sequence
  Oligonucleotide
aatagaagcttgtcgactgatctatccaaaactg

SEQ ID NO: 17 - DNA, 66 bp, Artificial Sequence
  Oligonucleotide
aaaagagctcggccagatcttctagaggatccaagaattctgttttatatttgttgtaaaaagtag

SEQ ID NO: 18 - DNA, 37 bp, Artificial Sequence
  Oligonucleotide
ttttgaattccaagatctcccatgtctctactggtgg

SEQ ID NO: 19 - DNA, 41 bp, Artificial Sequence
  Oligonucleotide
ccccgagctcgtcgacccttctcgaaagctttaacgaacgc

SEQ ID NO: 20 - DNA, 43 bp, Artificial Sequence
  Oligonucleotide
ttttgaattcaaagaatgagatttccttcaatttttactgcag

SEQ ID NO: 21 - DNA, 37 bp, Artificial Sequence
  Oligonucleotide
tttttctagactaggaggggtactcatactcctcggc

SEQ ID NO: 22 - DNA, 33 bp, Artificial Sequence
  Oligonucleotide
cgaatgtccatcgttgcgaacctgcagaacctg

SEQ ID NO: 23 - DNA, 33 bp, Artificial Sequence
  Oligonucleotide
caggttctgcaggttcctaacgatggacattcg

SEQ ID NO: 24 - DNA, 33 bp, Artificial Sequence
  Oligonucleotide
cgaatgtccatcgttaggaacctgcagaacctg

SEQ ID NO: 25 - DNA, 33 bp, Artificial Sequence; identical to SEQ ID NO: 23
  Oligonucleotide

SEQ ID NO: 26 - DNA, 18 bp, Artificial Sequence
  Oligonucleotide
tcgcagagaacggatggc

SEQ ID NO: 27 - DNA, 36 bp, Artificial Sequence
  Oligonucleotide
ttttgggcccttcatggtgatacggtatctcttggc

SEQ ID NO: 28 - DNA, 37 bp, Artificial Sequence
  Oligonucleotide
ttttctcgagaaggtggaacatactgccctgggatgg

SEQ ID NO: 29 - DNA, 38 bp, Artificial Sequence
  Oligonucleotide
ttttgagctcgtttaggaaacgtccttggcggagatgc

SEQ ID NO: 30 - DNA, 40 bp, Artificial Sequence
  Oligonucleotide
tttttctagacactgcgaatccatggtataaaccaaaacc

SEQ ID NO: 31 - DNA, 24 bp, Artificial Sequence
  Oligonucleotide
gtcgttgttcatggacatacctcc

SEQ ID NO: 32 - DNA, 26 bp, Artificial Sequence
  Oligonucleotide
tacaaatgttcttctgccatttctgg

SEQ ID NO: 33 - DNA, 32 bp, Artificial Sequence
  Oligonucleotide
ggttcatatgcgccggagctcctcgacagcag

SEQ ID NO: 34 - DNA, 37 bp, Artificial Sequence
  Oligonucleotide
ggttcctaggatccgcaagtttgattccattgcggtg

SEQ ID NO: 35 - DNA, 70 bp, Artificial Sequence
  Oligonucleotide
ttaaagagtaccttggctatagaataccgtagagataaagacctgaatagagattgtactgagagtgcac

SEQ ID NO: 36 - DNA, 70 bp, Artificial Sequence
  Oligonucleotide
aggtattataactatttttctgtattttttatatatttttatttgccaagctgtgcggtatttcacaccg

SEQ ID NO: 37 - DNA, 23 bp, Artificial Sequence
  Oligonucleotide
ctttggttaaagagtaccttggc

SEQ ID NO: 38 - DNA, 23 bp, Artificial Sequence
  Oligonucleotide
tactacgaaaagcgtgtgcgagg

SEQ ID NO: 39 - DNA, 71 bp, Artificial Sequence
  Oligonucleotide
tagaaggctactcaaaagaataaagttactataaaatatactgcggtatatagattgtactgagagtgcac

SEQ ID NO: 40 - DNA, 70 bp, Artificial Sequence
  Oligonucleotide
gatcggcaagaaactttgaagcagtatatttacaggattaaattatatatctgtgcggtatttcacaccg

SEQ ID NO: 41 - DNA, 22 bp, Artificial Sequence
  Oligonucleotide
cggaggggctctatgataaagg

SEQ ID NO: 42 - DNA, 23 bp, Artificial Sequence
  Oligonucleotide
gagtaactagggcttctcttccc

SEQ ID NO: 43 - protein, 85 aa, Homo sapiens
SGLQRAEEAPRRQLRVSQRTDGESRAHLGALLARYIQQARKAPSGRMSIVKNLQNLDPSHRISDRDYMGWMDFGRRSAEEYEYPS

SEQ ID NO: 44 - protein, 22 aa, Homo sapiens
QLRVSQRTDGESRAHLGALLAR

SEQ ID NO: 45 - protein, 19 aa, Homo sapiens
VSQRTDGESRAHLGALLAR

SEQ ID NO: 46 - protein, 51 aa, Homo sapiens
YIQQARKAPSGRMSIVKNLQNLDPSHRISDRDYMGWMDFGRRSAEEYEYPS

SEQ ID NO: 47 - protein, 17 aa, Homo sapiens
YIQQARKAPSGRMSIVK

SEQ ID NO: 48 - protein, 16 aa, Homo sapiens
YIQQARKAPSGRMSIV

SEQ ID NO: 49 - protein, 13 aa, Homo sapiens
NLQNLDPSHRISD

SEQ ID NO: 50 - protein, 23 aa, Homo sapiens
NLQNLDPSHRISDRDYMGWMDFG

SEQ ID NO: 51 - protein, 34 aa, Homo sapiens
NLQNLDPSHRISDRDYMGWMDFGRRSAEEYEYPS

SEQ ID NO: 52 - protein, 20 aa, Homo Sapiens
DYMGWMDFGRRSAEEYEYPS

SEQ ID NO: 53 - protein, 12 aa, Artificial Sequence
  Portion of fusion protein
KREAEASGLQRA

SEQ ID NO: 54 - protein, 9 aa, Homo sapiens
RMSIVKNLQ

SEQ ID NO: 55 - protein, 13 aa, Homo sapiens
DRDYMGWMDFGRR

SEQ ID NO: 56 - DNA, 48 bp, Artificial Sequence
  Oligonucleotide MFa1BNP (S)
ggataaaagagaggctgaagctcacccgctgggcagccccggttcagc

SEQ ID NO: 57 - DNA, 48 bp, Artificial Sequence
  MF1aBNP (AS)
gctgaaccggggctgcccagcgggtgagcttcagcctctcttttatcc

SEQ ID NO: 58 - DNA, 34 bp, Artificial Sequence
  Oligonucleotide BNP5′EcoRI
ttttgaattcatggatccccagacagcaccttcc

SEQ ID NO: 59 - DNA, 33 bp, Artificial Sequence
  Oligonucleotide BNP3′Xbal
tttttctagattaatgccgcctcagcactttgc

SEQ ID NO: 60 - DNA, 48 bp, Artificial Sequence; identical to SEQ ID NO: 56
  Oligonucleotide MF1BNP (S)

SEQ ID NO: 61 - DNA, 48 bp, Artificial Sequence; identical to SEQ ID NO: 57
  Oligonucleotide MF1BNP (AS)

SEQ ID NO: 62 - DNA, 69 bp, Artificial Sequence
  Oligonucleotide YPS15′GD400
aaaaagataaggtgaacaccaagcatatagtataatattacctaccacatgattgtactgagagtgcac

SEQ ID NO: 63 - DNA, 70 bp, Artificial Sequence
  Oligonucleotide YPS13′GD400
aactccaactggcttggagatgtgaatgtctaaactttgtgcaacggtttctgtgcggtatttcacaccg

SEQ ID NO: 64 - DNA, 20 bp, Artificial Sequence
  Oligonucleotide YPS15′DC
tcgtttcactgatgtgtccg

SEQ ID NO: 65 - DNA, 21 bp, Artificial Sequence
  Oligonucleotide YPS15′DC
gattataggccatatcccagg

SEQ ID NO: 66 - protein, 13 aa, Artificial Sequence
  Pitrilysin consensus sequence
GXXHXXEHXXXXG

SEQ ID NO: 67 - protein, 44 aa, Artificial Sequence
  Pitrilysin consensus sequence
GXXHXXEHXXXXGXXXXXXXXXXXXXXXXXXXXXXNAXTXXXXT

SEQ ID NO: 68 - protein, 44 aa, Artificial Sequence
  Pitrilysin consensus sequence
GXXHXXEHXXXXGXXKYPXXXXXXXXLXXXXXXXXNAXTXXXXT

(19) United States
(12) Patent Application Publication
(10) Pub. No.: US 2006/0121565 A1
(43) Pub. Date: Jun. 8, 2006

(54) Methods for increasing the production of a recombinant polypeptide from a host cell

(75) Inventors: Lars Jonson, Viby Sjaelland (DK); Jens F Rehfeld, Charlottenlund (DK); Anders H Johnsen, Roskilde (DK)

Correspondence Address: FOLEY AND LARDNER LLP;SUITE 500, 3000 K STREET NW, WASHINGTON, DC, 20007, US

(73) Assignee: CYMIP A/S

(21) Appl. No.: 10/528,563

(22) PCT Filed: Sep. 19, 2003
(86) PCT No.: PCT/DK03/00609

§ 371 Date: Oct. 11, 2005

(30) Foreign Application Priority Data
Sep. 20, 2002 (DK) PA200201391

(51) Int. Cl. C12P 21/06; C07H 21/04; C12N 9/64; C12N 15/85

(52) U.S. Cl. 435/69.1; 435/455; 435/226; 435/325; 536/23.2

(57) ABSTRACT

The present invention provides a host cell comprising a nucleic acid sequence encoding a recombinant polypeptide in which the production of a naturally occurring metalloprotease comprising a sequence provided in SEQ ID NO:1 has been reduced or inhibited by genetic manipulation. The present invention also relates to methods for enhancing the production of a polypeptide from a cell by disrupting the synthesis or activity of the metalloprotease. In particular, the present invention relates to methods for enhancing the secretion of recombinant polypeptides from host cells such as, but not limited to, yeast and bacterial cells. The metalloprotease is a member of the pitrilysin subfamily of proteases, characterized by comprising the sequence HXXEH (SEQ ID NO:1), where X is any amino acid.

### FIELD OF THE INVENTION

The present invention relates to methods for enhancing the production of a polypeptide from a cell by disrupting the synthesis or activity of a metalloprotease from the clan ME (M16 family). In particular, the present invention relates to methods for enhancing the secretion of recombinant polypeptides from host cells such as, but not limited to, yeast and bacterial cells.

### BACKGROUND OF THE INVENTION

Cholecystokinin (CCK) is a vertebrate neuroendocrine peptide hormone that is expressed in both gut and brain tissues. The maturation of bioactive CCK peptides depends on post-translational tyrosine sulfation, endoproteolytic cleavages, exoproteolytic trimmings and carboxyterminal amidation. The endoproteolytic processing of the N-terminus varies with CCK-83, -58, -39, -33, -22, -8 and -5 being identified. Most of the CCK peptides are synthesized after cleavage at a single Arg residue, however, CCK-22 requires processing after a single Lys residue.

Many recombinant polypeptides have been expressed in yeast as a fusion protein to the Saccharomyces cerevisiae α-factor prepro-peptide to direct secretion through the secretory pathway. The best characterized yeast protease is the serine endoprotease, Kex2p (Fuller et al., 1989) which is involved in maturation of the α-mating pheromone and of killer toxin (Julius et al., 1984). Another yeast protease is Yps1p belonging to the yapsin family of glycosyl-phophatidylinositol (GPI)-anchored aspartyl proteases, which is able to rescue mating deficiency when overexpressed in a kex2 mutant (Egel-Mitani et al., 1990). Expression of foreign proteins have shown that Yps1p and Yps2p contain endoprotease activity.

The use of host cells for the expression of recombinant polypeptides has greatly simplified the production of large quantities of commercially valuable polypeptides, which otherwise are obtainable only by purification from their native sources. There is a varied selection of expression systems currently available from which to choose for the production of any given polypeptide, including eubacterial and eukaryotic hosts. One important factor in the selection of an appropriate expression system is the ability of the host cell to produce adequate yields of the polypeptide. However, a problem frequently encountered is the high level of proteolytic enzymes produced by a given host cell or in the culture medium. Accordingly, there is a need for further methods which enhance the production of a recombinant polypeptide from a host cell.

Metalloproteases are the most diverse of the four main types of protease, with more than 30 families identified to date. In these enzymes, a divalent cation, usually zinc, activates the water molecule. The zinc metalloproteases can be divided based on the zinc binding site into for example Zincins and inverzincins (Hooper, N. M. 1994). The metal ion is held in place by amino acid ligands, usually three in number. The known metal ligands are His, Glu, Asp or Lys and at least one other residue which may play an electrophillic role is required for catalysis. Of the known metalloproteases, around half contain an HEXXH motif, which has been shown in crystallographic studies to form part of the metal-binding site.

A number of proteases dependent on divalent cations for their activity have been shown to belong to a single family, peptidase M16. Included are insulinase, mitochondrial processing protease, pitrilysin, nardilysin and a number of bacterial proteins. These proteins do not share many regions of sequence similarity; the most noticeable is in the N-terminal section. This region includes a conserved histidine followed, two residues later, by a glutamate and another histidine. In pitrilysin, it has been shown that this HXXEH motif is involved in enzymatic activity (Becker et al. 1992); the two histidines bind zinc and the glutamate is necessary for catalytic activity. The X can be any amino acid. Non active members of this family have lost from one to three of these active site residues.

It has previously been suggested that one could provide host cells and methods of producing proteins by expressing significantly reduced levels of a genetical modification in order to express significantly reduced levels of a metalloprotease containing an HEXXH motif in a filamentous fungal host cell, in e.g. U.S. Pat. No. 5,861,280 (WO 98/12300).

Others have provided a protease deficient filamentous fungus which is characterised in that the filamentous fungus contains a site selected disruption of DNA that results in the filamentous fungus having reduced metalloprotease activity and isolated DNA sequences encoding a protein having metalloprotease activity, which is obtainable from a filamentous fungus (WO 97/46689). Again this metalloprotease contains an HEXXH motif.

However, metalloproteases which can be reduced by a genetical modification in order to express significantly reduced levels of said metalloprotease in a non-filamentous fungal host cell and other cells containing an motif other than HEXXH have never been described.

### SUMMARY OF THE INVENTION

Whilst investigating the role various proteases play in processing proCCK in recombinant yeast, the present inventors surprisingly noted that deletion/disruption of CYM1 enhanced recombinant polypeptide production and secretion. Furthermore, the present inventors have found that Cym1p belongs to a family of metalloproteases, the activity of which can be down-regulated to enhance the levels of recombinant polypeptide produced from a host cell.

Accordingly, in a first aspect the present invention provides a host cell comprising a nucleic acid sequence encoding a recombinant polypeptide in which the production of a naturally occurring metalloprotease comprising a sequence provided in SEQ ID NO:1 has been reduced or inhibited by genetic manipulation.

The host cell can be any cell which, in its native state, possesses the metalloprotease. Accordingly, the host cell can be a eukaryotic or prokaryotic cell. Examples of preferred eukaryotic cells include, but are not limited to, mammalian cells, plants cells and fungal cells. In a preferred embodiment, the host cell is a yeast cell. More preferably, the yeast cell is selected from, but not limited to, the group consisting of: Saccharomyces sp. such as Saccharomyces cerevisiae, Saccharomyces paradoxus, Saccharomyces mikatae, Saccharomyces bayanus, Saccharomyces castelli and Saccharomyces kluyveri, Schizosaccharomyces sp. such as Schizosaccharomyces pombe, Kluyveromyces lactis, Hansenula polymorpha, Pichia pastors, Pichia methanolica, Pichia kluyveri, Yarrowia lipolytica, Candida utilis, Candida cacaoi, and Geotrichum fermentans.

Mettalloproteases are among the hydrolases in which the nucleophilic attack on a peptide bond is mediated by a water molecule. This is a characteristic shared with aspartic proteases, but in the metalloproteases a divalent metal cation, usually zinc, but sometimes cobolt or manganese, actives the water molecule. The metal ion is held in place by amino acid ligands usually 3 in number, the known metal ligands in metalloproteases are His, Glu, Asp or Lys residues.

Metalloproteases can be divided into two broad groups depending on the metal ions required for catalysis, and in the literature metalloproteases have been allocated into at least 8 different clans: MA, MB, MC, MD, ME, MF, MG and MH. Thus, illustrating the complex diversity of this group of proteases. The allocation is based on different consensus sequences due to the ligand binding, and thus each family have different biological substrates and/or functions.

The metalloproteases which are to be down regulated according to the present invention is a member of the pitrilysin subfamily (ME) of proteases, characterized by comprising the sequence HXXEH (SEQ ID NO:1) where X is any amino acid. Presently more than 180 members are annotated in Swissprot to the ME clan. Thus, the most preferred embodiment, the metalloprotease comprises a consensus sequence provided in SEQ ID NO:1. In another preferred embodiment, the metalloprotease comprises a consensus sequence provided in SEQ ID NO:2. Even more preferably, the metalloprotease comprises a consensus sequence provided in SEQ ID NO:3. In addition, it is preferred that the metalloprotease comprises SEQ ID NO:1 and a glutamic acid residue between 70 and 80 amino acids C-terminal of the second His residue. Further, it is preferred that the metalloprotease comprises a sequence selected from the group consisting of SEQ ID NO:1, SEQ ID NO:2 and SEQ ID NO:3 as well as a sequence selected from the group of:
i) any one of group consisting of SEQ ID NO's 4 to 15, and
ii) a sequence which is at least 80% identical to any one of SEQ ID NO's 4 to 15.

More preferably, the metalloprotease comprises SEQ ID NO:1 or SEQ ID NO:2 or SEQ ID NO:3 as well as a sequence selected from the group of:
i) any one of SEQ ID NO's 4 or 5, and
ii) a sequence which is at least 80% identical to any one of SEQ ID NO's 4 or 5.

Preferably, the metalloprotease comprises a sequence which is at least 85% identical, such as at least 90% identical, such as at least 95% Identical, and such as at least 99% identical to any one of SEQ ID NO's 4 to 15.

In a particularly preferred embodiment, the metalloprotease comprises a sequence as provided in SEQ ID NO:4, or a sequence at least 80% identical, such as at least 90%, such as at least 95% and such as 99% identical, thereto.

The host cell can be genetically manipulated by any means known in the art as long as the production of the metalloprotease is reduced or inhibited when compared to a parental host cell which has not been genetically manipulated. Such means of genetically manipulating the host cell include, but are not limited to; gene knockout, gene disruption, random or site directed mutagenesis, introduction of dominant-negative metalloproteases, RNA interference (RNAi) using dsRNA, catalytic nucleic acids (such as ribozymes and DNAzymes), and antisense nucleic acids. Preferably, the genetic manipulation acts directly upon the gene encoding the metalloprotease, the mRNA transcribed from the gene, or produces a protein that alters the activity of the metalloprotease such as a dominant negative mutant which competes with the metalloprotease for binding to a substrate but does not, for example, possess catalytic activity. However, the host cell may be genetically manipulated such that it indirectly affects the production or activity of the metalloprotease. For instance, the genetic manipulation can target a transcription factor involved in transcribing the mRNA encoded by the metalloprotease gene, thus at least reducing the levels of metalloprotease produced by the manipulated host cell.

Furthermore, the host cell may be further genetically manipulated such that it lacks at least one other naturally occurring protease of the host cell or has reduced activity for at least one other naturally occurring protease of the host cell. The protease can be any enzyme of which the inhibition increases the production of a recombinant polypeptide produced by the host cell. The protease can either be an endopeptidase, an aminopeptidase or a carboxypeptidase. Preferred proteases include serine proteases, aspartyl proteases, cysteine proteases and other metalloproteases.

In one embodiment, the host cell is a yeast cell and the other naturally occurring protease(s) is at least one protease encoded by any of the protease genes selected from the group consisting of; KEX2, YPS1 (previously known as YAP3), YPS2 (previously known as MKC7), YPS3, YPS6, YPS7, BAR1, STE13, KEX1, PRC1, PEP4 (also known as PRA1), APE1, APE2, APE3 and CPS1. Preferably, the host cell is a yeast cell and KEX2 production has been disrupted. Similar naturally occurring protease(s) within other host cells than yeast in addition to the metalloprotease specifically described here in could also be disrupted and/or genetically manipulated for an further additive enhancement.

The recombinant polypeptide can be any desired polypeptide which is capable of being produced in the host cell. The recombinant polypeptide can comprise a naturally occurring sequence or has been produced by the intervention of man (e.g. a mutant or truncation of a naturally occurring protein, or a fusion between at least two different polypeptides). Typically, the recombinant polypeptide will be of commercial value, for example in the treatment of diseases.

The recombinant polypeptide can be any size. Typically, the recombinant polypeptide will range in size from about 30 amino acids to about 4,500 amino acids. In one embodiment, the recombinant polypeptide is between about 30 to about 200 amino acids in length.

In at least some host cell expression systems for producing recombinant polypeptides, it is desirable to direct the recombinant polypeptide to be secreted from the host cell. Thus, in a preferred embodiment, the nucleic acid comprises a sequence which encodes a signal for directing the recombinant polypeptide to be secreted from the host cell. Preferably, the signal is an N-terminal hydrophobic signal sequence. Such N-terminal hydrophobic signal sequences are known in the art, and include, for example but not limited to, the leader sequence originating from the fungal amyloglucosidase (AG) gene such as galA—both 18 and 24 amino acid versions e.g. from Aspergillus sp., the α-factor gene such as yeasts e.g. from Saccharomyces sp. and Kluyveromyces sp., the P-factor of Schizosaccharomyces sp., and the α-amylase gene from Bacillus sp. In one embodiment, the recombinant polypeptide is expressed as a fusion of an N-terminal hydrophobic signal sequence and a second polypeptide sequence encoding the recombinant polypeptide which is from a different source than the signal sequence.

The nucleic acid encoding the recombinant polypeptide can be provided to the host cell using any technique known in the art. In one embodiment, the nucleic acid is inserted into the genome of the host cell using, for example, homologous recombination based techniques. In another embodiment, the nucleic acid is transfected or transformed into the host cell in an expression vector which remains extrachromosomal. For example, the expression vector can be a plasmid or a virus. Further, it is preferred that the vector comprises a selectable marker which can be used to selectively propagate host cells comprising the vector. Such selectable markers and the use thereof are also known in the art.

In a second aspect, the present invention provides a method of producing a recombinant polypeptide, the method comprising culturing a host cell according to the second aspect under suitable conditions such that the recombinant polypeptide is produced, and recovering the recombinant polypeptide.

Since the proteolytic action arising from the metalloprotease has been reduced or inhibited, the method of the second aspect of the invention improves the stability of the recombinant polypeptide produced by the host cell.

In a preferred embodiment, the recombinant polypeptide is secreted from the host cell. Furthermore, it is preferred that the secreted protein is recovered during exponential growth of a culture comprising the host cell.

Preferably, the quantity of the recovered recombinant polypeptide is higher than if a parental host cell was used. More preferably, the quantity of the recovered recombinant polypeptide is at 50% higher than if a parental host cell was used.

In a third aspect, the present invention provides a method of cleaving a polypeptide at a basic residue, the method comprising contacting the polypeptide, in the presence of a divalent cation, with a metalloprotease comprising a sequence selected from the group of:
i) any one of SEQ ID NO's 4 to 15, and
ii) a sequence which is at least 80% identical to any one of SEQ ID NO's 4 to 15.

In a particularly preferred embodiment, the metalloprotease comprises a sequence as provided in SEQ ID NO:4, or a sequence at least 80% Identical, such as at least 90%, such as at least 95% and such as 99% identical, thereto.

Preferably, the metalloprotease cleaves the polypeptide at the C-terminal side of an amino acid, or sequence of amino acids, selected from the group consisting of; Lys, Arg, ArgArg, LysLys, ArgLys and LysArg. Accordingly, it is preferred that the polypeptide comprises Lys, Arg, ArgArg, LysLys, ArgLys or LysArg. Other sequence requirements may also be necessary for cleavage, however, these can readily be determined by routine experimentation.

Preferably, the divalent cation is selected from the group consisting of: Zn²⁺, Co²⁺ and Mn²⁺.

The method of the third aspect can be performed in vivo, within a recombinant host cell producing the metalloprotease, or in vitro in suitable reaction conditions. Considering the present disclosure, the skilled addressee could readily perform the method of the third aspect. An example of an in vitro system for cleaving a polypeptide with the defined metalloprotease is provided herein. In this instance, the polypeptide is contacted with the metalloprotease provided in a crude yeast cell extract in 0.1 M NaH₂PO₄ (pH 4.5) and in the presence of 1 mM Mn²⁺ and 1 mM bestatin. In another example, the metalloprotease can be recombinantly produced as a fusion protein with a suitable “tag”, such as a His-tag, which enables easy purification of the fusion protein. Preferably, such a “tag” is removed (for example by enzymatic cleavage) before the metalloprotease is exposed to the substrate polypeptide.

In a fourth aspect, the present invention provides a method of identifying an agent that inhibits the activity of a metalloprotease comprising a sequence provided in SEQ ID NO:1, the method comprising the steps of:
a) incubating the metalloprotease with the agent, in the presence of a divalent cation and a suitable substrate;
b) determining the activity of the metalloprotease on the substrate;
c) comparing the activity obtained in step b) with the activity of a control sample that has not been incubated with the agent; and
d) selecting an agent that inhibits the activity of the metalloprotease.

The substrate can be any polypeptide that can be cleaved by the metalloprotease and the cleavage event detected. One example disclosed herein is the use of CCK as a substrate, where the cleavage event is detected by the production of CCK-22. Similar assays can readily be developed for other substrates.

In a preferred embodiment of the fourth aspect, the metalloprotease comprises a sequence as provided in SEQ ID NO:4, or a sequence at least 80% identical, such as at least 90%, such as at least 95% and such as 99% Identical, thereto.

In a fifth aspect, the present invention provides a method of producing a recombinant polypeptide, the method comprising culturing a host cell comprising a nucleic acid sequence encoding a recombinant polypeptide under suitable conditions such that the recombinant polypeptide is produced, and recovering the recombinant polypeptide wherein said culturing comprises the presence of an inhibitor of a metalloprotease comprising a sequence provided in SEQ ID NO:1.

Preferably, the inhibitor is identified according to a method of the fourth aspect.

As will be apparent, preferred features and characteristics of one aspect of the invention may be applicable to other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention will hereinafter be described by way of the following non-limiting Figures and Examples.

### BRIEF DESCRIPTION OF THE ACCOMPANYING DRAWINGS

FIG. 1. Cholecystokinin expression construct. PreproMfα1p-proCCK fusion protein with the amino acid sequences around the fusion site and of the primary cleavage sites shown. The major forms of secreted CCK with their N- and C-terminal amino acid residues are shown below.

FIG. 2. CCK-22 maturation in cells and media as a function of cell growth. BJ2168 expressing preproMfα1p-proCCK fusion protein. The CCK-22 immuno-reactivity was measured by RIA using Ab 89009 and total CCK content measured with Ab 89009 after tryptic cleavage. Open circles represent the fraction of secreted CCK-22, whereas the intracellular fraction of CCK-22 is presented as filled triangles. The cell growth was measured by OD₆₀₀ (open squares). The data represent mean of two independent experiments.

FIG. 3. Chromatographic analyses of normal and K→A mutated CCK secreted from BJ2168. Media from yeast transformed with pRS426 preproMfα1p-proCCK and pRS426 preproMfα1p-proCCK (K→A) were subjected to G-50 gel chromatography and the CCK immuno-reactivity was measured with Ab 7270 specific for Gly extended CCK (A and C) and Ab 89009, which is specific for the N-terminus of CCK-22 (B and D).

FIG. 4. In vitro protease assay including inhibitors and activators. The fraction of CCK-22 was calculated from the immuno-reactivity using Ab 89009 divided by the total amount of mature and N-terminal extended CCK-22 measured with Ab 89009 after trypsin treatment. A, Effect of different inhibitors. B, Protease reactivation by addition of 1.2 mM divalent metal ions to extracts where the activity had been inhibited with 1 mM EDTA. The data represent mean±SD of three independent experiments.

FIG. 5. Protease reactivation by Zn²⁺ and Mn²⁺. In vitro protease assays performed with cell extracts from LJY123, where the activity was inhibited with 1 mM EDTA (filled squares) and reactivated by addition of 1.2 mM Mn²⁺ (open circles) or 1.2 mM Zn²⁺ (filled circles). The activity was measured as the fraction of matured CCK-22 after 30, 60 and 120 min incubation. The fraction of CCK-22 was calculated from the immuno-reactivity using Ab 89009 divided by the total amount of mature and N-terminal extended CCK-22 measured with Ab 89009 after trypsin treatment. The data are represented by mean±SD (n=3).

FIG. 6. Extracellular CCK-22 maturation by members of the yapsin family. The ability of intact cells to process extracellular CCK was analysed as described under “Experimental Procedures” for BY4705 and the isogenic yps1, yps1 yps3 and yps1 yps2 yps3 strains. The fraction of CCK-22 was calculated from the immuno-reactivity using Ab 89009 divided by the total amount of total CCK measured with Ab 89009 after trypsin treatment. The data are represented by mean±SD (n=4). Statistics were performed using unpaired t test as described in experimental procedures (***=P<0.001, **=P<0.01 and *=P<0.05).

FIG. 7. Increased proteolysis following Cym1p overexpression. In vitro protease assays performed with cell extracts from BJ2168 transformed with an empty pRS425 plasmid (A) and with pRS425 containing CYM1 (B). The CCK-22 immuno-reactivity was measured over time using Ab 89009 (filled squares and circles) and the total amount of mature and N-terminal extended CCK-22 measured with Ab 89009 after trypsin treatment (open squares and circles). The data represent mean±SD of three independent experiments.

FIG. 8. Effects of KEX2 and CYM1 deletions on proCCK secretion and CCK-22 maturation. Yeast cells transformed with the proCCK expression construct were harvested during exponential phase and the media collected. The intracellular (A) and extracellular (B) amount of total CCK was measured with Ab 89009 after trypsin treatment. The fraction of intracellular (C) and secreted (D) CCK-22 was calculated as the immuno-reactivity measured with Ab 89009 before tryptic cleavage divided with the total amount of CCK measured in (A) and (B). The kex2, cym1 and kex2 cym1 strains are isogenic to BJ2168. The data are given as mean±SD (n=4). Statistics were performed using unpaired t test (***=P<0.001, **=P<0.01 and *=P<0.05). The stars enclosed in brackets are a comparison between the kex2 and kex2 cym1 strain.

FIG. 9. Intracellular degradation of CCK depends on Cym1p cleavage to CCK-22. Expression of wild type CCK, preproMfα1p-proCCK, and the CCK mutant, preproMfα1p-proCCK (K→A) in BJ2168 and a CYM1 disrupted strain isogenic to BJ2168. The cells were sedimented during exponential growth and the total amount of CCK (hatched bars) was measured after trypsin and carboxypeptidase B treatment with Ab 7270 specific for Gly-extended CCK. The amount of mature Gly-extended (white bars), which is dependent on translocation into the secretory pathway, Kex2p and carboxypeptidase activity is measured as the immuno-reactivity using Ab 7270 before tryptic cleavage and carboxypeptidase B treatment. The data are given as mean±SD (n=3).

FIG. 10. Aspartyl proteases involved in the maturation of CCK-22. Expression of wild type proCCK in BY4705 and the isogenic yapsin deletion strains of YPS1, YPS2 and YPS3. The intra-(A) and extracellular (B) fraction of synthesised CCK-22 was measured during exponential growth. The fraction of mature CCK-22 was calculated as the immuno-reactivity measured with Ab 89009 before tryptic cleavage divided with the total amount of CCK. The data are represented by mean±SD (n=3). Statistics were performed using unpaired t test as described in experimental procedures (***=P<0.001, **=P<0.01 and *=P<0.05).

FIG. 11. Cym1p processing C-terminally to both Lys and Arg residues. CYM1 deletion enhance the amount of secreted CCK more than two fold of both wild type CCK and the Lys⁶¹→Arg⁶¹ mutant. Expression of wild type CCK, preproMfα1p-proCCK, and the CCK mutant, preproMfα1p-proCCK (Lys⁶¹→Arg⁶¹) in BJ2168 and a CYM1 disruptant isogenic to BJ2168. Yeast cells were harvested during exponential phase and the media collected. The intracellular (A) and extracellular (B) amount of total CCK was measured with Ab 89009 after trypsin treatment. The data are given as mean±SD (n=3).

FIG. 12. Secreted proCCK fragments identified by mass spectrometry. The CCK-numbers refer to C-terminal amidated CCK. The molecular masses are given as monoisotopic values except for * which denote average value. Strain A, vacuolar protease-deficient strain (BJ2168), and B, the isogenic strain with KEX1 KEX2 disruptions (LJY22).

FIG. 13. Model for the production of the C-terminally extended CCK (A) and GLP2 (B). Expression of these fusion peptides should be performed in a sec61 mutant, or the pre-sequence of the α-mating factor should be removed to avoid translocation into the ER. The amino acid sequences around the fusion sites are shown. Underlined are the N- and C-terminal amino acids of the Gly-extended CCK-22 and GLP1.

FIG. 14

A. The preproMfα1p-proBNP expression construct.

B. The preproMfα1p-KREAEA-BNP-32 expression construct.

C. The preproMfα1p-KR-BNP-32 expression construct.

FIG. 15

A. The preproMfα1p-proBNP expression construct transformed in BJ2168, LJY430 (cym1 mutant), LJY431 (yps1 mutant) and LJY432 (cym1yps1 double mutant). Media was analysed from cells that have reached stationary phase using Ab 98192 that is specific for the N-terminus of proBNP. The cym1, yps1, and cym1yps1 strains are isogenic to BJ2168. The data are given as mean±SD (n=3). Statistics were performed using unpaired t test as described in experimental procedures (***=P<0.001, **=P<0.01 and *=P<0.05). The stars enclosed in brackets are comparisons are between the wild type strain, BJ2168 vs. cym1, BJ2168 vs. yps1 and yps1 vs. yps1cym1.

(ns,=not significant).

B. Analysis of proCCK fragments secreted from a cym1 mutant. Media containg 10 pmole proBNP was applied to Superdex 200 column on a Akta purifier system. The proBNP contant in the collected fractions were measured using Ab. 98192, that is specific for the N-terminus of proBNP.

### KEY TO THE SEQUENCE LISTING

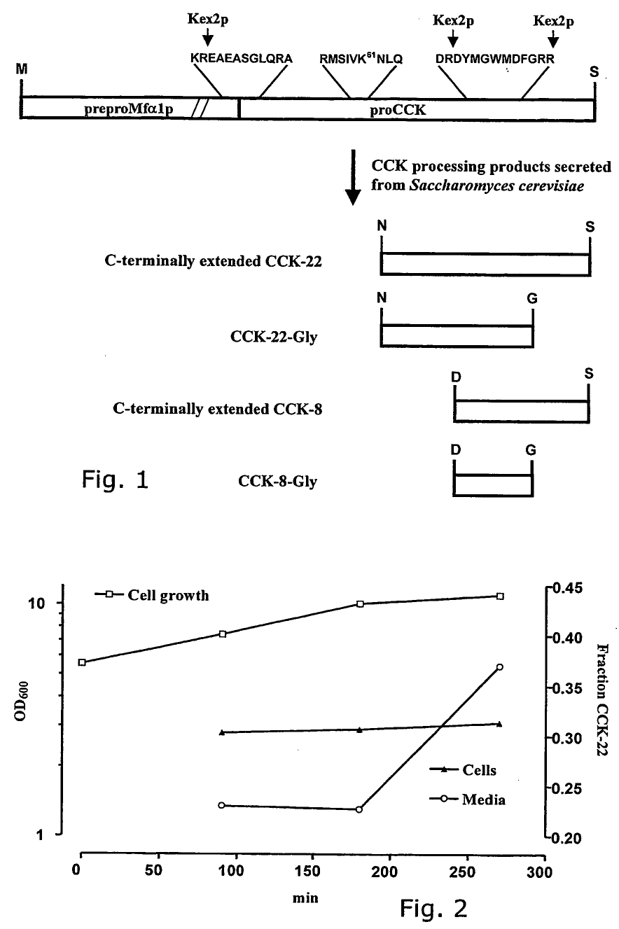

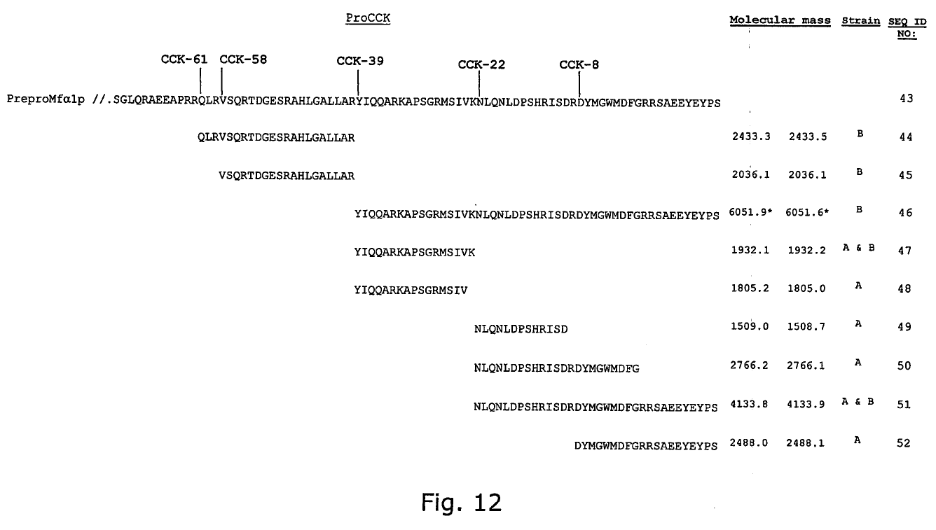

SEQ ID NO:1—Consensus sequence for pitrilysin proteases.
SEQ ID NO:2—Consensus sequence for at least some pitrilysin proteases.
SEQ ID NO:3—Consensus sequence for at least some pitrilysin proteases.
SEQ ID NO:4—Saccharomyces cerevisiae Cym1p (Swissprot Accession No. P32898).
SEQ ID NO:5—Schizosaccharomyces pombe C119.7 (Swissprot Accession No. O42908).
SEQ ID NO:6—Clostridium perfringens HypA protein (Swissprot Accession No. Q46205).
SEQ ID NO:7—Borrelia burgdorferi protein BB0228 (Swissprot Accession No. 051246).
SEQ ID NO:8—Caenorhabditis elegans C05D11.1 protein (Swissprot Accession No. P48053).
SEQ ID NO:9—E. coli protease III (Swissprot Accession No. P05458).
SEQ ID NO:10—Rat NRD convertase (Swissprot Accession No. P47245).
SEQ ID NO:11—Human insulysin (Swissprot Accession No. P14735).
SEQ ID NO:12—Arabidopsis thaliana CPE (Genbank Accession No. T03302).
SEQ ID NO:13—Human metalloprotease I (GenBank Accession No. AAH01150) in part, the full sequence (Swissprot Accession No. 095204).
SEQ ID NO:14—Bacillus subtilis zinc protease ymxG (GenBank Accession No. Q04805).
SEQ ID NO:15—Mycobacterium tuberculosis zinc protease Rv2782c (GenBank Accession No. O33324).
SEQ ID NO's 16 to 42—Oligonucleotides.
SEQ ID NO's 43 to 52—Sequences provided in FIG. 12.
SEQ ID NO's 53 to 55—Sequences provided in FIG. 1.
SEQ ID NO's 56 to 65—Oligonucleotides.
SEQ ID NO:66—Consensus sequence for at least some pitrilysin proteases.
SEQ ID NO:67—Consensus sequence for at least some pitrilysin proteases.
SEQ ID NO:68—Consensus sequence for at least some pitrilysin proteases.

### DETAILED DESCRIPTION OF THE INVENTION

The present invention provides a host cell useful for the expression of a polypeptide, said cell being genetically manipulated in order to at least produce reduced levels of a defined metalloprotease, when compared to the parental cell. The host cell will thus be able to express a protein of interest in higher quantity due to the proteolytic action of the metalloprotease has been reduced or inhibited which improves the stability of the protein of interest.

By the method of the invention, the proteolytic action of the metalloprotease has been reduced or inhibited, thereby improving the stability of the product obtained.

Thus, one embodiment of the present invention relates to a host cell useful for the expression of a protein of interest, wherein said cell has been genetically modified in order to express significantly reduced levels of a metalloprotease comprising a HXXEH motif (SEQ ID NO 1) compared to the corresponding non-modified cell when cultured under identical conditions.

The metalloproteases which are to be down regulated according to the present invention do not share many regions of sequence similarity; the most noticeable is in the N-terminal section. This region includes a conserved histidine followed two residues later by a glutamate and another histidine. In pitrilysin, it has been shown that this HXXEH motif is involved in enzymatic activity; the two histidines bind zinc and the glutamate is necessary for catalytic activity. Non active members of this family have lost from one to three of these active site residues.

The metalloprotease family which are to be down regulated according to the present Invention is presently classified as member of clan ME, family M16. This family is currently divided into 4 subfamilies:

M16A comprising pitrilysin

M16B comprising mitochondrial processing peptidase beta-subunit (Saccharomyces cerevisiae)

M16C comprising eupltrilysin (Homo sapiens)

M44 comprising vaccinia virus-type metalloindopeptidase (vaccinia virus).

Sequence alignments of these proteins show several sequence similarities. These sequence similarities are highly conserved and can be used to distinguish members of this family from non-members.

Among such sequence similarities several individual amino acids are highly conserved and are easily recognisable in specific positions navigated from the HXXEH motif.

Thus, one embodiment of the present invention relates to a host cell, wherein the metalloprotease comprises a glutamic acid residue between 70 and 80 amino acids C-terminal of the second His residue in the HXXEH motif.

A further embodiment of the present invention relates to a host cell, wherein the metalloprotease comprises a glysine residue 3 amino acids N-terminal of the first His residue in the HXXEH motif.

Another embodiment of the present invention relates to a host cell, wherein the metalloprotease comprises a glysine residue 5 amino acids C-terminal of the second His residue in the HXXEH motif.

One further embodiment of the present invention relates to a host cell wherein the metalloprotease comprises a lysine residue 8 amino acids C-terminal of the second His residue in the HXXEH motif.

Also, one embodiment of the present invention relates to a host cell, wherein the metalloprotease comprises a tyrosine residue 9 amino acids C-terminal of the second His residue in the HXXEH motif.

Furthermore, the present invention relates to a host cell, wherein the metalloprotease comprises a proline residue 10 amino acids C-terminal of the second His residue in the HXXEH motif.

Among the sequence similarities several regions of amino acids are also highly conserved and are easily recognised. Thus, in a presently preferred embodiment the invention relates to a host cell wherein the metalloprotease comprises the consensus sequence SEQ ID NO 2.

In another presently preferred embodiment, the invention relates to a host cell, wherein the metalloprotease comprises the consensus sequence SEQ ID NO 3.

In a presently most preferred embodiment, the invention relates to a host cell, wherein the metalloprotease comprises a NAXTXXXXT motif between 20 and 30 amino acids C-terminal of the second His residue in the HXXEH motif.

In a presently another preferred embodiment, the invention relates to a host cell, wherein the metalloprotease comprises the consensus sequence SEQ ID NO 66-68. One embodiment of the present invention relates to a host cell useful for the expression of a protein of interest, wherein said cell has been genetically modified in order to express significantly reduced levels of a metalloprotease which is at least 80% identical to the any of SEQ ID NO: 4-15, as compared to a parental cell.

In the present context, the term “protein of interest” relates to any of the numerous naturally native occurring extremely complex substances such as but not limited to proteins, enzymes and/or antibodies that consist of amino acid residues joined by peptide bonds. It is an object of preferred embodiments of the present invention to provide such native proteins which are products of the host cell itself and/or heterologous proteins, fusion proteins, recombinant proteins, eukaryotic proteins, prokaryotic proteins, lysosomal proteins, vacuolar proteins, precursor proteins, zymogene proteins, prepro-proteins, and secreted proteins.

Preferrred embodiments of the claimed method are advantageous due to the higher production of the protein of interest, thus any increase of the amount of the protein of interest when produced in a host cell modified as described herein compared to the amount produced in the corresponding non-modified cell when cultured under identical conditions are within the scope of the present invention.

One assay in which a skilled addressee could evaluate enhanced production of the protein of interest in a host cell modified as described here in and compared to the amount produced in the corresponding non-modified cell, is by culturing the two different host cells under identical condition, and measure the amount of produced protein of interest by radio-immune assay using an antibody specific for the protein of interest. One such assay is describe in more detail in the examples of the present description.

One embodiment of the present invention relates to a host cell, wherein the total amount of the protein of interest is increased at least 5% compared to the corresponding non-modified cell when cultured under identical conditions, such as at least 10% compared to the corresponding non-modified cell when cultured under identical conditions, such as at least 20% compared to the corresponding non-modified cell when cultured under identical conditions, such as at least 50% compared to the corresponding non-modified cell when cultured under identical conditions, such as at least 100% compared to the corresponding non-modified cell when cultured under identical conditions, such as at least 200% compared to the corresponding non-modified cell when cultured under identical conditions, or even at least 1000% or compared to the corresponding non-modified cell when cultured under identical conditions.

In the present context, the term “host cell” relates to any cell capable of producing the protein of interest. Thus, in one prefered embodiment, the host is a prokaryotic cell. In another preferred embodiment, the host cell is a eukaryotic cell, such as but not limited to a filamentous fungal cell and a non-filamentous fungal cell. Non limiting examples hereof are a strain of Saccharomyces, especially Saccharomyces cerevisiae.

All the features described herein relating to the methods of the present invention are also applicable as embodiments relating to the host cells, and vice versa.

The method described in the present application relates to the production of a protein of interest in a host cell, wherein said host cell has been genetically modified in order to express significantly reduced levels of a metalloprotease which is at least 80% identical to the SEQ ID NO: 4 as compared to a parental cell, when cultured under identical conditions, comprising
a) introducing into the host cell a nucleic acid sequence encoding the protein of interest;
b) cultivating the host cell of step (a) in a suitable growth medium for production of the protein of interest and
c) isolating the protein of interest.

One embodiment of the present invention relates to a method for the production of a protein of interest in a host cell, wherein the host cell has been genetically modified by a method selected from the group comprising gene knock-out, gene disruption, random or site directed mutagenesis, introduction of dominant-negative metalloproteases, RNA Interference (RNAi) using dsRNA, catalytic nucleic acids (such as ribozymes and DNAzymes), antisense nucleic acids or a combination thereof.

In a presently most preferred embodiment, the host cell is essentially free of any metalloprotease activity.

One preferred embodiment of the present invention relates to a method for the production of a protein of interest in a host cell, in which the protein of interest is a eukaryotic protein, selected from the group comprising insulin, growth hormone, glucagon, somatostatin, interferon, adrenocorticotropic hormones, angiotensinogen, atrial natriuretic peptides, dynorphin, endorphines, galanin, gastrin, gastrin releasing peptides, neuropeptide Y fragments, pancreastatin, pancreatic polypeptides, secretin, vasoactive intestinal peptide, growth hormone releasing factor, melanocyte stimulating hormone, neurotensin, adrenal peptide, parathyroid hormone and related peptides, somatostatin and related peptides, brain natriuretic peptide, calcitonin, corticotropin releasing factor (CRF), cocaine amphetamine regulated transcript (CART), thymosin, urotensin, glucagon and glucagon like peptides (GLP-1 and GLP-2), somatostatin, interferon, a vascular endothelial growth factor (VEGF), platelet derived growth factor (PDGF), factor VII, factor VIII, factor V, factor IX, interleukins, urokinase, erythropoietin (EPO), chymosin, tissue plasminogen activator, Positive cofactor 2 glutamine/Q-rich-associated protein (PCAP), peptide tyrosine tyrosine (PYY), ghrelin, orexin, Beta-neoendorphin-dynorphin precursor, CCK or serum albumin.

Another preferred embodiment of the present invention relates to a method for the production of a protein of interest in a host cell, in which the protein of interest is a protein of fungal origin, selected from the group comprising an amylolytic enzyme, an alpha-amylase, a beta-amylase, a glyco-amylase, a alpha-galactosidase, a cellulytic enzyme, a lipolytic enzyme, a xylanolytic enzyme, a proteolytic enzyme, an oxidoreductase, a peroxidase, a laccase, a pectinase, or a cutinase.

A further preferred embodiment of the present invention relates to a method for the production of a protein of interest in a host cell, in which the protein of interest is a bacterial protein, selected from the group comprising an amylolytic enzyme, an alpha-amylase, a beta-amylase, a glyco-amylase, a beta-galactosidase, a cellulytic enzyme, a lipolytic enzyme, a xylanolytic enzyme, a proteolytic enzyme, an oxidoreductase, a peroxidase, a laccase, a pectinase, or a cutinase.

A special embodiment of the present invention relates to a method for production of a protein of interest in a host cell, in which the protein of interest is a precursor, i.e. a zymogen, a hybrid protein, a protein obtained as a pro sequence or pre-pro sequence, or in unmaturated form.

General Molecular Biology

Unless otherwise indicated, the recombinant DNA techniques utilised in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), F. M. Ausubel et al. (Editors), Current Protocols in Molecular Biology, Greene Pub. Associates and Wiley-Interscience (1988, including all updates until present), Methods in Enzymology. Vol 194. Guide to Yeast Genetics and Molecular Biology. (1991) Ed Gunthrie and Fink Academic Press, Methods in Microbiology Vol. 26. Yeast Gene Analysis. (1998) Ed. Brown and Tuite. Academic Press, Miller, I. H. (1992) A Short Course in Bacterial Genetics (Manual, L., ed), Cold Spring Harbor Laboratory, Cold Spring Harbor, N.Y., Johnston, J. R (1994) Molecular Genetics of Yeast (A Practical Approach) Oxford University Press, Oxford., and Molecular Genetics of Yeast: A Practical Approach, Ed. J. R. Johnston, IRL Press (1994) and are incorporated herein by reference.

The % identity of a polypeptide is determined by GAP (Needleman and Wunsch, 1970) analysis (GCG program) with a gap creation penalty=5, and a gap extension penalty=0.3. The query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. Even more preferably, the query sequence is at least 100 amino acids in length and the GAP analysis aligns the two sequences over a region of at least 100 amino acids. More preferably, the query sequence is at least 250 amino acids in length and the GAP analysis aligns the two sequences over a region of at least 250 amino acids. Even more preferably, the query sequence is at least 500 amino acids in length and the GAP analysis aligns the two sequences over a region of at least 500 amino acids.

Pitrilysin Subfamily of Metalloproteases

The pitrilysin subfamily of metalloproteases is characterized by the presence of a HXXEH (SEQ ID NO:1) motif. A general review of this subfamily is provided by Rawlings and Barrett (1995). Members of this family include, but are not limited to, S. cerevisiae Cym1p (SEQ ID NO:4) (Swissprot Accession No. P32898), Schizosaccharomyces pombe C119.7 (SEQ ID NO:5) (Swissprot Accession No. O42908), Clostridium perfringens HypA protein (SEQ ID NO:6) (Swissprot Accession No. Q46205), Borrelia burgdorferi protein BB0228 (SEQ ID NO:7) (Swissprot Accession No. 051246), Caenorhabditis elegans C05D11.1 protein (SEQ ID NO:8) (Swissprot Accession No. P48053), E. coli protease III (also known as pitrilysin) (SEQ ID NO:9) (Swissprot Accession No. P05458), rat NRD convertase (SEQ ID NO:10) (Swissprot Accession No. P47245), human insulysin (SEQ ID NO:11) (Swissprot Accession No. P14735), Arabidopsis thaliana CPE (SEQ ID NO:12) (Genbank Accession No. T03302), human metalloprotease I (in part) (SEQ ID NO:13) (GenBank Accession No. AAH01150) (the full sequence: Swissprot Accession No. 095204), Bacillus subtilis zinc protease ymxG (SEQ ID NO:14) (GenBank Accession No. Q04805), and Mycobacterium tuberculosis zinc protease Rv2782c (SEQ ID NO:15) (GenBank Accession No. O33324). For E. coli protease III (SEQ ID NO:9) it has been shown that the His residues of SEQ ID NO:1, as well as Glu-169, are involved in divalent cation binding whilst the Glu residue flanked by the His residues is a catalytic residue.

A gene encoding a pitrilysin metalloprotease can readily be identified by screening by hybridization for nucleic acid sequences coding for all of, or part of, the metalloprotease, e.g. by using synthetic oligonucleotide probes, that may be prepared on the basis of a cDNA sequence, e.g. the nucleotide sequences encoding any one of the metalloproteases presented as SEQ ID NO's: 4 to 15, in accordance with standard techniques.

Genetic Manipulations

The host cell of the invention which is genetically manipulated in order to produce reduced levels of the defined metalloprotease may be modified using standard recombinant DNA technology known to the person skilled in the art. The gene sequence responsible for the production of the metalloprotease may be inactivated or eliminated entirely.

In a particular embodiment, the host cell of the invention is one genetically manipulated at the coding or regulatory regions of the metalloprotease gene. Known and useful techniques include, but are not limited to, gene knockout, gene disruption, random or site directed mutagenesis, introduction of dominant-negative metalloproteases, RNA Interference (RNAi) using dsRNA, catalytic nucleic acids (such as ribozymes and DNAzymes), and antisense nucleic acids, or a combination thereof.

Mutagenesis may be performed using a suitable physical or chemical mutagenizing agent. Examples of a physical or chemical mutagenizing agent suitable for the present purpose includes ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulfite, formic acid, and nucleotide analogues. When such agents are used, the mutagenesis is typically performed by incubating the cell to be mutagenized in the presence of the mutagenizing agent of choice under suitable conditions for the mutagenesis to take place, and selecting for mutated cells having a significantly reduced production of metalloprotease.

Genetic manipulation may also be accomplished by the introduction, substitution or removal of one or more nucleotides in the metalloprotease coding sequence or a regulatory element required for the transcription or translation thereof. Nucleotides may, for example, be inserted or removed so as to result in the introduction of a stop codon, the removal of the start codon or a change of the open reading frame. The modification or inactivation of the structural sequence or a regulatory element may be accomplished by site-directed mutagenesis or PCR generated mutagenesis in accordance with methods known in the art.

A convenient way to inactivate or reduce the metalloprotease production of a host cell is based on the principles of gene interruption. This method involves the use of a DNA sequence corresponding to the endogenous gene or gene fragment which it is desired to destroy. The DNA sequence is in vitro mutated to a defective gene and transformed into the host cell. By homologous recombination, the defective gene replaces the endogenous gene or gene fragment. It may be desirable that the defective gene or gene fragment encodes a marker which may be used for selection of transformants in which gene encoding the metalloprotease has been modified or destroyed.

The term “antisense” as used herein refers to nucleotide sequences which are complementary to a specific nucleic acid sequence. Antisense molecules may be produced by any method, including synthesis by ligating the gene(s) of interest in a reverse orientation to a viral promoter which permits the synthesis of a complementary strand. Once introduced into a host cell, this transcribed strand combines with natural sequences, in this instance that encoding the metalloprotease, produced by the cell to form duplexes. These duplexes then block either the further transcription or translation. In this manner, mutant phenotypes may be generated.

The term “catalytic nucleic acid” refers to a DNA molecule or DNA-containing molecule (also known in the art as a “deoxyribozyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T and U, as well as derivatives thereof. Derivatives of these bases are well known in the art.

Typically, the catalytic nucleic acid contains an antisense sequence for specific recognition of a target nucleic acid, and a nucleic acid cleaving enzymatic activity, also referred to herein as the “catalytic domain”. The types of ribozymes that are particularly useful in this invention are the hammerhead ribozyme (Haseloff and Gerlach, 1988) and the hairpin ribozyme (Shippy et al., 1999).

Ribozymes useful for the methods of the invention, and DNA encoding the ribozymes, can be chemically synthesized using methods well known in the art. The ribozymes can also be prepared from a DNA molecule (that upon transcription yields an RNA molecule) operably linked to an RNA polymerase promoter, e.g., the promoter for T7 RNA polymerase or SP6 RNA polymerase. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase. Alternatively, the ribozyme can be modified to the phosphothio analog for use in liposome delivery systems. This modification also renders the ribozyme resistant to endonuclease activity.

dsRNA (RNAi) is particularly useful for specifically inhibiting the production of a particular protein. This technology relies on the presence of dsRNA molecules that contain a sequence that is essentially identical to the mRNA of the gene of interest, in this case a mRNA encoding the metalloprotease. Conveniently, the dsRNA is produced in a single open reading frame in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for genetic manipulation is well known within the capacity of a person skilled in the art, particularly considering Waterhouse et al. (1998), Elbashir et al. (2001), WO 99/32619, WO 99/53050, WO 99/49029, and WO 01/34815.

Owing to the genetic manipulation, the host cell of the invention expresses significantly reduced levels of the metalloprotease. In a preferred embodiment, the level of metalloprotease expressed by the host cell is reduced more than about 25%, such as more than about 30%, such as more than about 35%, such as more than about 40%, such as more than about 45%, such as more than about 50%, such as more than about 55%, such as more than about 60%, such as more than about 65%, such as more than about 70%, such as more than about 75%, such as more than about 80%, such as more than about 85%, such as more than about 90%, such as more than about 95%, such as more than about 98%, and such as more than about 99%.

In a presently most preferred embodiment, the product expressed by the host cell is essentially free of any activity of the defined metalloprotease.

In the present context, the term “essentially free” relates to a host, wherein the metalloprotease expressed by said host cell is reduced to a level, where the function of said metalloprotease has no biologically significant reducing influence on the production of the protein of interest.

Protein of Interest

The terms “polypeptide”, “protein” and “peptide” are used herein interchangeably and in the present context relates to any of the numerous naturally occurring extremely complex substances such as but not limited to enzymes or antibodies that consist of amino acid residues joined by peptide bonds, contain the elements carbon, hydrogen, nitrogen, oxygen, usually sulphur, and occasionally other elements such as but not limited to phosphorus or iron, that are essential constituents of all living cells, that are in nature synthesised from raw materials by plants but assimilated as separate amino acids by animals, that are both acidic and basic and usually colloidal in nature although many have been crystallised, and that are hydrolyzable by acids, alkalies, proteolytic enzymes, and putrefactive bacteria to polypeptides, to simpler peptides, and ultimately to alpha-amino acids.

As defined herein, a “recombinant polypeptide” is a protein which is not native to the host cell, or a native polypeptide in which modifications have been made to alter the native sequence, or a native protein whose expression is quantitatively altered as a result of a manipulation of a native regulatory sequence required for the expression of the native protein, such as a promoter, a ribosome binding site, etc., or other manipulation of the host cell by recombinant DNA techniques.

Owing to the absence or reduction in activity of the defined metalloprotease, at least a portion of the recombinant polypeptides expressed by the host cell may also be a precursor protein, i.e. a zymogen, a hybrid protein, a protein obtained as a pro sequence or pre-pro sequence, or in unmaturated form.

In a more specific embodiment, the recombinant polypeptide is of eukaryotic origin, such as insulin, adrenocorticotropic hormones, angiotensinogen, atrial natriuretic peptides, dynorphin, endorphines, galanin, gastrin, gastrin releasing peptides, neuropeptide Y fragments, pancreastatin, pancreatic polypeptides, secretin, vasoactive intestinal peptide, growth hormone releasing factor, melanocyte stimulating hormone, neurotensin, adrenal peptide, parathyroid hormone and related peptides, somatostatin and related peptides, brain natriuretic peptide, calcitonin, corticotropin releasing factor (CRF), cocaine amphetamine regulated transcript (CART), thymosin, urotensin, glucagon and glucagon like peptides (GLP-1 and GLP-2), somatostatin, interferon, a vascular endothelial growth factor (VEGF), platelet derived growth factor (PDGF), factor VII, factor VIII, factor V, factor IX, interleukins, urokinase, erythropoletin (EPO), chymosin, tissue plasminogen activator, CCK or serum albumin.

With specific regard to “glucagon and glucagon like peptides”, this term as used herein may refer to polypeptides of human origin or from other animals and recombinant or semisynthetic sources and include all members of the glucagon family, such as GRPP (glicentine related polypeptide), glucagon, GLP-1 (glucagon like peptide 1), and GLP-2 (glucagon like peptide 2), including truncated and/or N-terminally extended forms, such as GLP-1(7-36), and includes analogues, such as GLP-1(7-35)R36A GLP-2 F22Y, GLP-2 A19T+34Y. GLP2 A2G and GLP-2 A19T, and other analogues having from 1 to 3 amino acid changes, additions and/or deletions.

Host Cells and the Expression of Recombinant Polypeptides Therefrom

The host cells for use in the present invention can be prokaryotic or eukaryotic. The eukaryotic host cells for use in the present invention can be, for example, fungal, mammalian, plant or insect cells. Preferably, the host cells are yeast cells.

In order to produce the desired polypeptide, the host cell of the invention comprises a nucleic acid sequence encoding the recombinant polypeptide as well as regulatory sequences for directing the expression of the desired product such as regions comprising nucleotide sequences necessary or e.g. transcription, translation and termination. The genetic design of the host cell of the invention may be accomplished by the person skilled in the art, using standard recombinant DNA technology for the transformation or transfection of a host cell.

Preferably, the host cell is modified by methods known in the art for the introduction of an appropriate expression cassette in, for example a plasmid or a viral vector, comprising the nucleic acid encoding the recombinant polypeptide. The expression cassette may be introduced into the host cell by a number of techniques including, but not limited to, as an autonomously replicating plasmid or integrated into the chromosome.

Expression cassettes may contain regulatory sequences such as transcription control sequences, translation control sequences, origins of replication, and other regulatory sequences that are compatible with the recombinant cell and that control the expression of nucleic acid molecules encoding the recombinant polypeptide. In particular, recombinant nucleic acid molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, arthropod and mammalian cells, such as, but not limited to, tac, lac, trp, trc, oxy-pro, omp/lpp, rrnB, bacteriophage lambda, bacteriophage T7, T7lac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Hellothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells. Transcription control sequences of the present invention are most preferably naturally occurring transcription control sequences associated with yeast. Suitable promoters for S. cerevisiae include the MFα1 promoter, galactose inducible promoters such as GAL1, GAL7 and GAL10 promoters, glycolytic enzyme promoters including TPI1 and PGK1 promoters, TRP1 promoter, CYCI promoter, CUP1 promoter, PHOS promoter, ADH1 promoter, and HSP promoter. A suitable promoter in the genus Pichia is the AOXI (methanol utilisation) promoter.

Recombinant polypeptides of the present invention may also (a) contain secretory signals to enable an expressed polypeptide to be secreted from the cell that produces the polypeptide and/or (b) contain fusion sequences which lead to the expression of fusion proteins. Examples of suitable signal segments include any signal segment capable of directing the secretion of the fusion protein. Preferred signal segments include, but are not limited to, tissue plasminogen activator (t-PA), interferon, Interleukin, growth hormone, histocompatibility and viral envelope glycoprotein signal segments, the leader sequence originating from the fungal amyloglucosidase (AG) gene such as galA—both 18 and 24 amino acid versions e.g. from Aspergillus sp., the α-factor gene of yeasts e.g. from Saccharomyces sp. and Kluyveromyces sp., the P-factor of Schizosaccharomyces sp., and the α-amylase gene from Bacillus sp., as well as natural signal sequences.

The cloning vehicle may also comprise a selectable marker, e.g. a gene, the product of which complements a defect in the host cell, or one which confers antibiotic resistance, such as ampicillin, kanamycin, chloramphenicol or tetracycline resistance.

The procedures used to ligate the DNA construct of the invention, the promoter, terminator and other elements, respectively, and to insert them into suitable cloning vehicles containing the information necessary for replication, are well known to persons skilled in the art.

Recombinant DNA technologies can be used to improve the expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules useful for the methods of the present invention include, but are not limited to, operably linking the nucleic acid molecule to high-copy number plasmids, integration of the nucleic acid molecule into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecule to correspond to the codon usage of the host cell, and the deletion of sequences that destabilize transcripts. The activity of an expressed recombinant polypeptide of the present invention may be improved by fragmenting, modifying, or derivatizing polynucleotide molecules encoding such a protein.

Methods of Producing Recombinant Polypeptides

Host cells that have been transfected or transformed with the nucleic acid encoding the recombinant polypeptide are cultured for a sufficient time and under appropriate conditions known to those skilled in the art in view of the teachings disclosed herein to permit the production, and preferably secretion, of the polypeptide, followed by recovery of the desired product.

Furthermore, owing to the reduced activity of the metalloprotease, the recombinant polypeptide expressed by the host cell may be obtained as a precursor protein, i.e. a zymogen, a hybrid protein, a protein obtained as a pro sequence or pre-pro sequence, or in unmaturated form.

The broth or medium used for culturing may be any conventional medium suitable for growing the host cell in question, and may be composed according to the principles of the prior art. The medium preferably contains carbon and nitrogen sources and other inorganic salts. Suitable media, e.g. minimal or complex media, are available from commercial suppliers, or may be prepared according to published protocols.

With regard to yeast host cells, it is often advantageous to produce heterologous polypeptides in a diploid yeast culture, because possible genetical defects may become phenotypically expressed in a haploid yeast culture, e.g. during continuous fermentation in production scale, and because the yield may be higher. The production of recombinant polypeptides in yeast host cell is described in Molecular Genetics of Yeast: A Practical Approach, Ed. J. R. Johnston, IRL Press (1994) which is incorporated herein by reference.

After cultivation, the protein is recovered by conventional methods for isolation and purification proteins from a culture broth. Well-known purification procedures include separating the cells from the medium by centrifugation or filtration, precipitating proteinaceous components of the medium by means of a salt such as ammonium sulphate, and chromatographic methods such as e.g. ion exchange chromatography, gel filtration chromatography, affinity chromatography, etc.

The present invention is exemplified by demonstrating that the total amount of CCK and proBNP is increased about 60% about 100%, respectively when compared to the non-modified host cell. The examples thus demonstrate that the host cells of the present invention are able to increase the production of diverse proteins.

Further, the examples disclose that additional disruption of other proteases enhance the production of the protein of interest, for example disruption of both KEX2 and CYM1 results in an additive effect in the yield in the production of CCK (nearly 100%).

Further, it will be understood by the skilled addressee that special amino acids within some of the motifs described, particularly His, Glu, Asp or Lys, are essential to the function of the metalloprotease by functioning as metal ligands.

Furthermore, it will be recognised that the metalloproteases of the present invention have been annotated widely in the literature as family members of the pitrilysin family, insulysin family, insulinase family, inverzincin family and M16 subfamily of clan ME.

Thus, it will be understood that any feature and/or aspect discussed above in connection with any of these different family annotations apply by analogy to the metalloprotease described herein, which all include the HXXEH motif.

However, please note that pitrilysin (without family) in itself refers to a specific member of clan ME of metalloproteases in E. coli.

### EXAMPLES

Materials and Methods

Yeast Strains and Growth Conditions

The yeast strains used are listed in Table I. Construction of strains were carried out using either the two step gene disruption technique (Rothstein, 1991) or the PCR based method by (Brachmann et al., 1998). Media were purchased from Difco, amino acids and other supplements from Sigma-Aldrich. Yeast cells were grown at 30° C. in YPD (1% yeast extract, 2% peptone and 2% dextrose) or synthetic complete media (SC) based on yeast nitrogen base with ammonium sulfate, succinic acid, NaOH and appropriate amino acids. Transformations with either linear DNA or plasmids were performed using the modified lithium acetate procedure as described (Gletz et al., 1995). Analysis of heterologous expressed CCK was performed from yeast growing in exponential phase due to the consistency in CCK-22 biosynthesis, in contrast to the results from yeast within the stationary phase (FIG. 2). ProCCK processing was analysed from cell extract and media of 5 A₆₀₀ units of cells per ml synthetic complete media. Cell growth was followed by the absorbance at 600 nM.

TABLE 1
S. cerevisiae strains used in this study. Null mutants
of putative metalloproteases are named by the ORF in the
genotype and (*) represents mitochondrial proteases.

| Strain | Genotype | Source |
|---|---|---|
| BY4705 | MATα ade2Δ::hisG his3Δ200 | (Brachmann |
| | leu2Δ0 lys2Δ0 ura3Δ0 | et al., 1998) |
| LJY13 | MATα ade2Δ::hisG his3Δ200 | This study |
| | leu2Δ0 lys2Δ0 ura3Δ0 yps1::TRP1 | |
| LJY14 | MATα ade2Δ::hisG his3Δ200 | This study |
| | leu2Δ0 lys2Δ0 ura3Δ0 yps1::TRP1 | |
| | yps3::LEU2 | |
| LJY15 | MATα ade2Δ::hisG his3Δ200 | This study |
| | leu2Δ0 lys2Δ0 ura3Δ0 yps1::TRP1 | |
| | yps3::LEU2 yps2::URA3 | |
| BJ2168 | MATa prc1-407 prb1-1122 pep4-3 leu2 trp1 | (Jones, 1991) |
| | ura3-52 gal2 | |
| LJY21 | MATa prc1-407 prb1-1122 pep4-3 leu2 trp1 | This study |
| | ura3-52 gal2 kex1::LEU2 | |
| LJY22 | MATa prc1-407 prb1-1122 pep4-3 leu2 trp1 | This study |
| | ura3-52 gal2 kex1::LEU2 kex2::TRP1 | |
| LJY23 | MATa prc1-407 prb1-1122 pep4-3 leu2 trp1 | This study |
| | ura3-52 gal2 kex2::TRP1 | |
| LJY122 | MATα ape1::KANMX ape2::LYS2 his3Δ0 | This study |
| | leu2Δ0 lys2Δ0 ura3Δ0 | |
| LJY123 | MATα ape1::KANMX ape2::LYS2 | This study |
| | ape3::LEU2 his3Δ0 leu2Δ0 | |
| | lys2Δ0 ura3Δ0 | |
| LJY201 | MATa prc1-407 prb1-1122 pep4-3 leu2 | This study |
| | trp1 ura3-52 gal2 axl1::LEU2 | |
| LJY202 | MATa prc1-407 prb1-1122 pep4-3 leu2 | This study |
| | trp1 ura3-52 gal2 ste24::LEU2 | |
| LJY203 | MATa prc1-407 prb1-1122 pep4-3 leu2 | This study |
| | trp1 ura3-52 gal2 prd1:LEU2 | |
| LJY204 | MATa prc1-407 prb1-1122 pep4-3 leu2 | This study |
| | trp1 ura3-52 gal2 yil108w::LEU2 | |
| Y15298 | MATα his3Δ1 leu2Δ0 lys2Δ0 | Euroscarf |
| | ura3Δ0 ste23::KANMX | |
| Y11874 | MATα his3Δ1 leu2Δ0 lys2Δ0 | Euroscarf |
| | ura3Δ0 aap1::KANMX | |
| Y10148 | MATα his3Δ1 leu2Δ0 lys2Δ0 | Euroscarf |
| (*) | ura3Δ0 afg3::KANMX | |
| Y14953 | MATα his3Δ1 leu2Δ0 lys2Δ0 | Euroscarf |
| | ura3Δ0 ape1::KANMX | |
| Y16224 | MATα his3Δ1 leu2Δ0 lys2Δ0 | Euroscarf |
| (*) | ura3Δ0 rca1::KANMX | |
| Y14984 | MATα his3Δ1 leu2Δ0 lys2Δ0 | Euroscarf |
| (*) | ura3Δ0 mip1::KANMX | |
| Y17144 | MATα his3Δ1 leu2Δ0 lys2Δ0 | Euroscarf |
| | ura3Δ0 yme1::KANMX | |
| Y13211 | MATα his3Δ1 leu2Δ0 lys2Δ0 | Euroscarf |
| | ura3Δ0 ybr074w::KANMX | |
| Y13801 | MATα his3Δ1 leu2Δ0 lys2Δ0 | Euroscarf |
| | ura3Δ0 ydl104c::KANMX | |
| Y11941 | MATα his3Δ1 leu2Δ0 lys2Δ0 | Euroscarf |
| | ura3Δ0 yhr113w::KANMX | |
| Y11960 | MATα his3Δ1 leu2Δ0 lys2Δ0 | Euroscarf |
| | ura3Δ0 yhr132c::KANMX | |
| Y12296 | MATα his3Δ1 leu2Δ0 lys2Δ0 | Euroscarf |
| | ura3Δ0 yil137c::KANMX | |
| Y15370 | MATα his3Δ1 leu2Δ0 lys2Δ0 | Euroscarf |
| | ura3Δ0 ynl045w::KANMX | |
| 10864B | MATα ura3-Δ851 leu2-Δ1 | Euroscarf |
| | his3Δ200 lys2Δ202 | |
| | ykr035c-ykr038c::URA3 | |
| Y11749 | MATα his3Δ1 leu2Δ0 lys2Δ0 | Euroscarf |
| | ura3Δ0 yol057w::KANMX | |
| Y16248 | MATα his3Δ1 leu2Δ0 lys2Δ0 | Euroscarf |
| | ura3Δ0 yol098c::KANMX | |
| 10231B | MATα his3Δ200 leu2Δ1 | Euroscarf |
| | trp1Δ63 ura3-52 | |
| | yol154w(4,744)::KANMX | |
| Y14266 | MATα his3Δ1 leu2Δ0 lys2Δ0 | Euroscarf |
| | ura3Δ0 ydr430c::KANMX | |
| LJY430 | MATa prc1-407 prb1-1122 pep4-3 leu2 | This study |
| | trp1 ura3-52 gal2 ydr430c::LEU2 | |
| LJY432 | MATa prc1-407 prb1-1122 pep4-3 leu2 | This study |
| | trp1 ura3-52 gal2 kex2::TRP1 | |
| | ydr430c::LEU2 | |

DNA Extraction and Amplification

Yeast genomic DNA was isolated as described (Philippsen et al., 1991). Polymerase chain reaction (PCR) was performed using either Pwo polymerase or the enzyme cocktail based on Taq, Pwo and Pfu polymerase (Expand long range PCR kit, XL-PCR) both from Roche. All PCR products were visualised by agarose gel-electrophoresis and PCR products either purified from the gel using the gel-extraction kit (Qiagen) or from the reaction mixture by PCR purification spin columns (GENOMED). PCR based one step gene disruption was performed using 50 ng of plasmid from the pRS400 series (Brachmann et al., 1998) as template. Amplification of the marker was performed with oligonucleotides having 20 nucleotides towards the plasmid and additional 50 nucleotides flanking the target gene (Table 2). All other DNA manipulations were carried out according to standard procedures (Sambrook et al., 1989).

Plasmid Constructions

Expression of proCCK was performed in pRS426 [2μ URA3] (Brachmann et al., 1998) using the phosphoglycerate kinase promoter (PGK1p) and terminator (PGK1t). The PGK1 promoter was amplified with PGK1p 5′HindIII and PGK1p 3′MCS (Table 2) using 100 ng of genomic yeast DNA as template and subsequently cloned into pGEM-11 (Promega) in the HindIII and SacI restriction enzyme sites. The terminator was amplified with PGK1t 5′Bg/II and PGK1t 3′SacI (Table 2) and ligated into the plasmid containing the promoter at the SacI and EcoRI restriction enzyme sites. This construct, pGEM-11 PGK1pMCSPGK1t then contained the PGK1-promoter, a multiple cloning site (MCS) with the restriction enzyme sites EcoRI, BamHI, XbaI and Bg/II followed by the PGK1 terminator. The preproMfα1p-proCCK fusion (Rourke et al., 1997) (FIG. 1) was subcloned into the EcoRI and XbaI sites of pGEM-11 PGK1pMCSPGK1t and finally the entire gene was cloned into pRS423 as well as pRS426 to complete the yeast CCK expression constructs, pRS423 preproMfα1p-proCCK and pRS426 preproMfα1p-proCCK respectively. Expression of CYM1 on a multi copy plasmid was constructed by amplification of the open reading frame (ORF) of CYM1 and additional 926 bp at the 5′ end and 703 bp at the 3′ end. The amplification was carried out by XL-PCR using 100 ng of genomic yeast DNA and the oligonucleotides, CYM1 5′ApaI and

TABLE 2
Oligonucleotides used.

| | | |
|---|---|---|
| Oligo | Oligonucleotide sequence (5′-3′) | Purpose |

| | | |
|---|---|---|
| PGK1p5′HindIII | AATAGAAGCTTGTCGACTGATCTATCCAAAACTG | Expression |
| | (SEQ ID NO: 16) | construct |
| PGK1p3′MCS | AAAAGAGCTCGGCCAGATCTTCTAGAGGATCCA | Expression |
| | AGAATTCTGTTTTATATTTGTTGTAAAAAGTAG | construct |
| | (SEQ ID NO: 17) | |
| PGK1t5′Bg/II | TTTTGAATTCCAAGATCTCCCATGTCTCTACTG | Expression |
| | GTGG (SEQ ID NO: 18) | construct |
| PGK1t3′SacI | CCCCGAGCTCGTCGACCCTTCTCGAAAGCTTTA | Expression |
| | ACGAACGC (SEQ ID NO: 19) | construct |
| 5′MFα1-EcoRI | TTTTGAATTCAAAGAATGAGATTTCCTTCAATT | preproMfα1p- |
| | TTTTACTGGAG (SEQ ID NO: 20) | proC CK |
| CCK3′-XbaI | TTTTTCTAGACTAGGAGGGGTACTCATACTCCT | preproMfα1p- |
| | CGGC (SEQ ID NO: 21) | proC CK |
| CCK-22 K→A(S) | CGAATGTCCATCGTTGCGAACCTGCAGAACCTG | Lys⁶¹→Ala⁶¹ |
| | (SEQ ID NO: 22) | mutation |
| CCK-22 K→A(AS) | CAGGTTCTGCAGGTTCCTAACGATGGACATTCG | Lys⁶¹→Ala⁶¹ |
| | (SEQ ID NO: 23) | mutation |
| CCK-22 K→R(S) | CGAATGTCCATCGTTAGGAACCTGCAGAACCTG | Lys⁶¹→Arg⁶¹ |
| | (SEQ ID NO: 24) | mutation |
| CCK-22 K→R(AS) | CAGGTTCTGCAGGTTCCTAACGATGGACATTCG | Lys⁶¹→Arg⁶¹ |
| | (SEQ ID NO: 25) | mutation |
| CCK-22 seq | TCGCAGAGAACGGATGGC (SEQ ID NO: 26) | Sequencing |
| CYM15′ApaI | TTTTGGGCCCTTCATGGTGATACGGTATCTCTT | Cloning of |
| | GGC (SEQ ID NO: 27) | CYM1 |
| CYM13′XhoI | TTTTCTCGAGAAGGTGGAACATACTGCCCTGGG | Cloning of |
| | ATGG (SEQ ID NO: 28) | CYM1 |
| KEX25′ | TTTTGAGCTCGTTTAGGAAACGTCCTTGGCGGA | Cloning of |
| | GATGC (SEQ ID NO: 29) | KEX2 |
| KEX23′ | TTTTTCTAGACACTGCGAATCCATGGTATAAAC | Cloning of |
| | CAAAACC (SEQ ID NO: 30) | KEX2 |
| KEX2DC5′ | GTCGTTGTTCATGGACATACCTCC | Control of |
| | (SEQ ID NO: 31) | Δkex2 |
| KEX2DC3′ | TACAAATGTTCTTCTGCCATTTCTGG | Control of |
| | (SEQ ID NO: 32) | Δkex2 |
| TRP15′NdeI | GGTTCATATGCGCCGGAGCTCCTCGACAGCAG | Cloning of |
| | (SEQ ID NO: 33) | TRP1 |
| TRP13′AvrII | GGTTCCTAGGATCCGCAAGTTTGATTCCATTGC | Cloning of |
| | GGTG (SEQ ID NO: 34) | TRP1 |
| KEX15GD400 | TTAAAGAGTACCTTGGCTATAGAATACCGTAGA | KEX1 |
| | GATAAAGACCTGAATAGAGATTGTACTGAGAGT | deletion |
| | GCAC (SEQ ID NO: 35) | |
| KEX13′GD400 | AGGTATTATAACTATTTTTCTGTATTTTTTATA | KEX1 |
| | TATTTTTATTTGCCAAGCTGTGCGGTATTTCAC | deletion |
| | ACCG (SEQ ID NO: 36) | |
| KEX15′DC400 | CTTTGGTTAAAGAGTACCTTGGC | Control of |
| | (SEQ ID NO: 37) | Δkex1 |
| KEX13′DC400 | TACTACGAAAAGCGTGTGCGAGG | Control of |
| | (SEQ ID NO: 38) | Δkex1 |
| CYM15′GD400 | TAGAAGGCTACTCAAAAGAATAAAGTTACTATA | CYM1 |
| | AAATATACTGCGGTATATAGATTGTACTGAGAG | deletion |
| | TGCAC (SEQ ID NO: 39) | |
| CYM13′GD400 | GATCGGCAAGAAACTTTGAAGCAGTATATTTAC | CYM1 |
| | AGGATTAAATTATATATCTGTGCGGTATTTCAC | deletion |
| | ACCG (SEQ ID NO: 40) | |
| CYM15′DC400 | CGGAGGGGCTCTATGATAAAGG | Control of |
| | (SEQ ID NO: 41) | Δcym1 |
| CYM13′DC400 | GAGTAACTAGGGCTTCTCTTCCC | Control of |
| | (SEQ ID NO: 42) | Δcym1 |

CYM13′XhoI (Table 2). The PCR product was purified on spin columns and subsequently cloned into the ApaI and XhoI restriction enzyme sites of pRS425.

The Lys⁶¹ residue, believed to be crucial for the proteolysis of proCCK to release CCK-22, was exchanged by Ala by site-directed mutagenesis (Horton et al., 1993). The exchange was performed by PCR using the Pwo polymerase (Boehringer Mannheim), where two products were amplified with the oligonucleotides sets, PGK1p5′ HindIII/CCK-22 K→A (antisense) and CCK-22 K→A (sense)/PGK1t3′ SacI (Table 2) and 50 ng of pRS426 preproMfα1p-proCCK as template to each reaction. The two products were subjected to agarose gel-electrophoresis and approximately 1 mm² of each product where cut out and used directly as template in a third PCR reaction. In this reaction the full-length cDNA encoding the fusion protein was amplified using PGK1p5′ HindIII and PGK1t3′ SacI (Table 2). The PCR product was subcloned into pCR-Blunt II (Invitrogen) and sequenced with the CCK specific primer, CCK-22 seq. Finally the PGK1p preproMfα1p-proCCK (K→A) PGK1t product was cloned into the HindIII and SacI sites of pRS426 to construct the expression plasmid, proCCK (K→A). Substitution of Lys with Arg was performed as described above by exchanging the CCK specific primers with CCK-22 K→R (antisense) and CCK-22 K→R (sense) to construct the proCCK (K→R) vector.

Strain Construction

Construction of a partial KEX2 disruption was performed in BJ2168 by amplification of the entire KEX2 gene with 1000 bp on each site of the ORF by XL-PCR using 100 ng of genomic yeast DNA and the oligonucleotides, KEX2 5′SacI and KEX2 3′XbaI (Table 2). The PCR product was purified on-spin columns and cloned into pCR-Blunt II (Invitrogen). Amplification of TRP1 was performed by XL-PCR introducing an NdeI site 925 bp 5′ to the ORF and an AvrII site 212 bp 3′ to the stop codon using TRP1 5′NdeI and TRP1 3′AvrII (Table 2). The PCR product was purified and subcloned into the NdeI and AvrII sites of KEX2 eliminating 2018 bp of KEX2 and 170 bp of the promoter. The kex2::TRP1 construct was excised from pCR-Blunt II using the NotI and SpeI restriction enzymes and subsequently transformed into BJ2168. Transformants were selected on SC-Trp plates followed by a colony PCR screen to test for correct integration using oligonucleotides that cover the entire marker plus an additional 1200 bp on each site of KEX2, kex2 DC5′ and kex2 DC3′ (Table 2). Construction of a kex2 kex1 strain was performed by the two step gene disruption technique (Rothstein, 1991) using the LEU2 marker. Amplification of LEU2 was performed by XL-PCR using 50 ng of pRS405 as template and kex15′GD400 and kex13′GD400 (Table 2). The PCR product was purified using PCR purification spin kit (GENOMED) and subsequently transformed into LJY23. Transformants were selected on SC-Leu plates and correct integration was tested by PCR-based colony screen using kex15′DC and kex13′DC (Table 2).

A Δcym1::LEU2 (LJY430) strain in a BJ2168 background was constructed by the one step gene disruption technique as described above for the Δkex1 strain using the oligonucleotides, CYM15′GD400 and CYM13′GD400 (Table 2) for gene disruption and cym15′DC and cym13′DC (Table 2) for disruption control. All null mutants created by this method were prepared with oligonucleotides designed towards the 50 bases adjacent to the 5′ and 3′ UTR with a specific 3′ end to the pRS400 series of vectors containing various markers (Brachmann et al., 1998). Transformants were selected on appropriate agar plates followed by a colony PCR screen to test for correct integration using oligonucleotides that cover the entire marker plus an additional 200 bp on each site. Only the oligonucleotides that are not positioned as described above are shown in Table 2.

Gene deletions of STE24, AXL1, PRD1 and YIL108w were made in BJ2168 using the PCR disruption technique (Brachmann et al., 1998) and pRS405 [LEU2] as template.

The LJY123, which contain gene deletions of APE1, -2 and -3, was derived from Y14953 using PCR disruption technique (Brachmann et al., 1998). APE2 was initially replaced with the LYS2 (pRS317 [cen; LYS2]) where the PCR product was purified from agarose gel prior to transformation and APE3 was substituted with the LEU2 marker (pRS405 [LEU2]).

The yps1 yps2 yps3 triple mutant (LJY15) was constructed in BY4705 using the PCR disruption technique (Brachmann et al., 1998). The ORF of YPS1 were initially deleted by insertion of the TRP1 locus (pRS404) to generate LJY13. This strain was then used as host for the deletion of YPS3 by insertion of the LEU2 marker (pRS405) and finally the YPS2 was deleted by insertion of the URA3 marker by amplification of pRS406 [URA3] to construct LJY15 (Table I).

CCK and CYM1 Expression

Human proCCK was expressed as a fusion protein between the prepro leader sequence of yeast α-mating factor and proCCK (preproMfα1p-proCCK). The fusion construct was expressed on multi-copy plasmids, with constitutively gene transcription from the phosphoglycerate kinase promoter. “ProCCK expression” refers to expression using pRS426 preproMfα1p-proCCK, which was used in all yeast strains with exception of BY4705 and the isogenic yapsins deletion strains, where proCCK was expressed from pRS423 preproMfα1p-proCCK. CYM1 expression was driven by its own promoter. Plasmid constructs, and oligonucleotides used are listed in Table 2.

Enzymatic Treatment

Trypsin treatment was performed using 1 mg/ml Trypsin (Worthington Biochemical Corporation) in a 50 mM sodium phosphate buffer (pH 7.5) for 30 min at RT and terminated by immersion into boiling water for 10 min. Carboxypeptidase B (Boehringer Mannheim) treatment with a final concentration of 4 μg/ml was performed in 0.1 mM sodium phosphate buffer (pH 7.5) at room temperature for 30 min. The reaction was terminated by immersion into boiling water for 10 min.

Gel Chromatography

Yeast transformants grown to late exponential phase were centrifuged at 15000 g to collect the cells and 500 μl of the medium was loaded directly onto a Sephadex G-50 superfine (Pharmacia) column 1×100 cm) at 4° C. The sample was eluted in VBA buffer (20 mM barbital buffer, 0.11% bovine serum albumin and 0.6 mM thiomersal) at a flow rate of 3.5 ml/h and fractions were collected every 17 min. Calibrations were performed by including ¹²⁵I-albumin (V₀) and ²²NaCl (Vₜ). The elution constants K_{d}, of peaks eluting at Vₑ are calculated as K_{d}=(Vₑ−V₀)/(Vₜ−V₀).

Radio-Immunoassay

Two different antisera were used to determine the amount of processed cholecystokinin. Ab 89009 (Paloheimo et al., 1994) is specific for the N-terminus of CCK-22 and Ab 7270 (Hilsted et al., 1986) is specific for Gly-extended CCK. The fraction of CCK processed to CCK-22 is calculated by division of the immuno-reactivity measured with Ab 89009 with the amount measured with the same antibody after the sample was treated with trypsin to measure the total amount of N-terminal extended CCK-22.

Yeast Extract and Protease Assay

Ten A₆₀₀ units of yeast cells growing in exponential phase were sedimented by centrifugation at 3000 g for 5 min, washed once in 25 ml H₂O and transferred to a 2 ml Eppendorf tube. An equal amount of acid washed glass beads (Sigma-Aldrich) was added followed by 200 μl of 0.1 M NaH₂PO₄ (pH 4.5) including various inhibitors (150 μM Bestatin, 30 μM E-64, 10 μM Leupeptin, 1 μM Pepstatin A, 1 mM phenylmethylsulfonyl fluoride (PMSF), 1 mM EDTA and 1 mM 1,10-orthophenanthrollne or 1 tablet complete inhibitor with or without EDTA per 2.5 ml 0.1 M NaH₂PO₄ (Boehringer Mannheim)). The cells were broken by vortexing 3×20 sec and the extracts were clarified by centrifugation at 15000 g for 10 min. All steps were carried out at 4° C. The protease assay was performed using 20 pmol synthetic amidated CCK-33 (Peninsula Laboratorie Europe, Merseyside, England) or Ac-CCK-33-Gly (Cambridge Research Biochemicals, Stockton, England) as substrate, 20 μl yeast extract, various inhibitors and activators in a total volume of 30 μl. The mixture was incubated at 30° C. for 1 h and the reaction terminated by adding 500 μl VBA buffer followed by immediate immersion into a boiling water bath for 10 min.

Protease Assay Using Metalloprotease Deficient Strains

The assay was performed as described above, but with addition of 1 mM Bestatin and 1 mM Mn²⁺ to decrease N-terminal degradation.

Protease Assay Using Intact Yeast Cells

Five A₆₀₀ units of exponential growing cells where sedimented, washed once in 5 ml H₂O and once in SC media (pH 6.0), before the cells where resuspended in 25 μl SC media. The protease assay was performed by addition of 20 pmol synthetic Ac-CCK-33-Gly as substrate and the mixture incubated with gentle shaking at 30° C. for 1 h. The reaction was terminated by addition of 500 μl VBA and the cells removed by centrifugation before the supernatant was immersed into boiling water for 10 min.

Analysis of Secreted CCK by MALDI-TOF

Fifty A₆₀₀ units of CCK transformed yeast cells were subjected to 25 ml of fresh media, followed by inoculation for 3 h. Cells were removed by centrifugation at 15000 g for 10 min and 500 μl of media was concentrated and desalted by reverse phase using a ZipTip C₁₈ column (Millipore). The peptides were eluted with 10 μl 50% acetonitrile. The purified peptides were analysed in a Matrix Assisted Laser Desorption/Ionization time-of-flight mass spectrometer (Biflex, Bruker-Franzen, Bremen, Germany) operated in the reflected mode using time lag focusing (delayed extraction). For analysis, 0.5 μl of the sample was mixed with 0.5 μl matrix solution (α-cyano-4-hydroxycinnamic acid in acetonitrile/methanol, Hewlett Packard). Then 0.5 μl of the mixture was applied to the probe and allowed to dry before introduction into the mass spectrometer.

Statistical Analysis

Statistical calculations were performed using an unpaired students t-test to analyse whether the change in proCCK expression or the fraction of mature CCK-22 between wild type yeast expressing proCCK and mutants isogenic to the wild type strain can be considered to be statistically significant. The P-value calculated for CCK-22 processing between yapsin mutants are comparisons of BY4705 and each mutant, whereas the brackets represent comparisons between BY4705 yps1 and BY4705 yps1 yps3 and, BY4705 yps1 yps3 and BY4705 yps1 yps2 yps3, respectively.

Expression of proBNP in Saccharomyces Cerevisiae—Construction of the yps1 Mutant

Cloning of preproBNP

Messenger RNA was isolated from a 500 mg Biopsy from human heart using the Quickprep Micro mRNA purification Kit (Amersham Pharmacia Biotech). First strand cDNA was prepared from 2 μg mRNA in a reaction containing, 2.5 μl 10× first strand buffer (Promega), 2.5 μl 100 mM DTT, 2.5 μl 10 mM dNTP, 2.5 μl Na pyrophosphate, 10 pmol Oligo(dT)₁₈, 10 units reverse transcriptase, AMV (Promega), and H₂O to 25 μl. Messenger RNA and Oligo(dT)₁₈ was heated to 70° C. for 5 min cooled on ice for 5 min prior to cDNA synthesis. The first strand cDNA syntesis was performed at 42° C. for 60 min.

The cDNA encoding preproBNP was ampified using Pwo polymerase (Roche), 1 μl 1. strand cDNA, 5 μl 10×Pwo buffer included MgCl₂ (Roche), 5 μl 2.5 mM dNTP, 30 pmol of each primer (BNP5′EcoRI and BNP3′XbaI). The PCR product encoding preproBNP was cloned in pBluescript II (Stratagene). All subsequent PCR reactions were performed as described above.

The fusion between the cDNA's encoding the preprosequence of the α-mating factor and proBNP was performed using overhang extension PCR, where two PCR reactions were set up. One using 50 ng of pRS426 preproMfα1p-proCCK as template, MFα15′EcoR1 and MF1BNP (AS) as primers and a second with 50 ng of preproBNP cloned in pBluescript and the primers, MFLBNP (S) and BNP3′XbaI. In the third PCR reaction, approximately 50 ng of each PCR products were purified from agarosegel from the the two initial PCR using the gel-extraction kit (Qiagen) and used as template with the two oligonucleotides, MFα15′EcoRI and BNP3′XbaI. The preproMfα1p-proBNP encoding construct was subcloned in pCR-Blunt II (Invitrogen) and sequenced with vector specific oligonucleotides prior to subcloning into the EcoRI and XbaI sites of pGEM-11 PGK1pMCSPGK1t. Finally the entire gene was cloned into pRS426 to complete the yeast proBNP expression constructs, pRS426 preproMfα1p-proBNP.

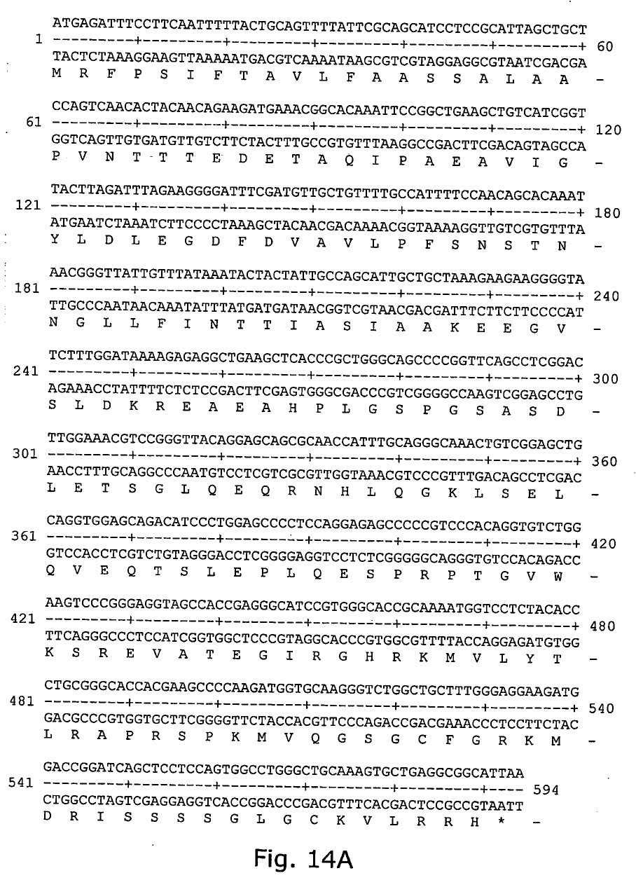

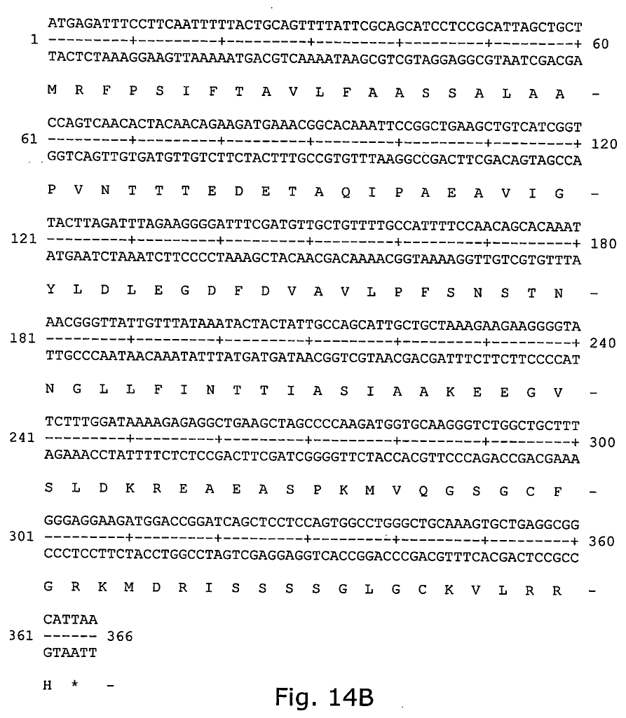

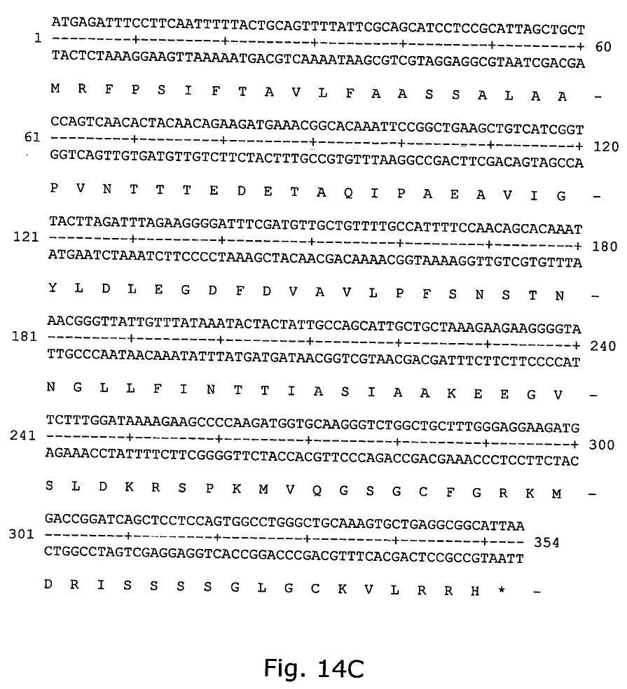

Furthermore, two additional constructs have been made, in which the proBNP fragment (1-76) has been removed. These constructs are similar to do the preproMfα1p-proBNP, but do only synthesise BNP-32. In the first construct, the Kex2p cleavage site and the spacer peptide of the preproMfα1p has been sustained (KREAEA)(FIG. 14B), whereas in the other construct, the spacer peptide has been removed (FIG. 14C). Analysis of the BNP-32 expression from wild type yeast and the Isogenic CYM1 disruptant will be analysed by RIA's using the Shionoria-BNP system from Electra-Box Diagnostica ApS. This assay is specific for BNP-32.

Expression of proCCK, proBNP and Cym1p

Human proCCK was expressed as a fusion protein between the prepro leader sequence of yeast α-mating factor and proCCK (preproMfα1p-proCCK). The fusion construct was expressed on multi-copy plasmids, with constitutively gene transcription from the phosphoglycerate kinase promoter. “ProCCK expression” refers to expression using pRS426 preproMfα1p-proCCK, which was used in all yeast strains with exception of BY4705 and the isogenic yapsins deletion strains, where proCCK was expressed from pRS423 preproMfα1p-proCCK. Human proBNP was also expressed as a fusion protein between the prepro leader sequence of yeast α-mating factor and proBNP (preproMfα1p-proBNP) (FIG. 14A). CYM1 expression was driven by its own promoter. Plasmid constructs, and oligonucleotides used are listed in Table 2.

BNP Radioimmunoassay

Antibody 98192 is specific for the N-terminus of proBNP (Gøtze et al., 2002).

Chromatography

FPLC chromatography was performed on a Superdex 200 column on a Äkta purifier (Amersham Pharmacia Biotech). In the 50 mM Na-phospate buffer, 100 mM NaCl and 6 M Guanidin were included.

Strain Construction

A Δyps1::TRP1 (LJY440) and a Δcym1::LEU2 Δyps1::TRP1 (LJY431) strain in a BJ2168 background were constructed by the one step gene disruption technique as described above using the oligonucleotides, YPS15′GD400 and YPS13′GD400 (Table 2) for gene disruption. The PCR product was transformed into BJ2168 and LJY430. Verification of the correct integration of the disruption cassete was analysed by PCR using yps15′DC and yps13′DC (Table 2).

Identification of the Gene Encoding the Cym1 Orthologue in Pichia Pastoris or Pichia Methanolica

Identification of the unknown genes from Pichia pastoris and Pichia methanolica encoding the Cym1p orthologues of Saccharomyces cerevisiae will be carried out in similar manner, using the same set of degenerated primers mentioned below. Pichia pastoris and Pichia methanolica will be refered to as Pichia in the following text.

By alignment of the orthologous Cym1 proteins of Saccharomyces kluveri and Schizosaccharomyces pombe to Cym1p from Saccharomyces cerevisiae, there was identified a number of identical amino acid sequences. From these sequences it is possible to syntesize degenerated oligonucleotides (Table 3) that will bind to the complementary DNA strands of CYM1 in all three species, and thus to the CYM1 gene of Pichia. Amplification of the genomic sequence will initially be carried out by using high quality genomic DNA as template, Pichia-CYM1-Ia and Pichia-CYM1-Ib and the Pwo polymerase (Roche). The amplified sequence with an expected size of approximately 525 bp will be cloned in pBlunt or a similar vector and sequenced with vector specific primers. If no band appear from the initial amplification, a second round of PCR will be performed with the two nested primers, Pichia-CYM1-IIa and Pichia-CYM1-IIb using 1 μl of the first PCR reaction as template. The expected product is approximately 270 bp and will be cloned in pBlunt and sequenced with M13 foreward and M13 reverse primers. From the obtained sequence there will be synthesized sequence specific primers, two nested sense and two nested antisense specific primers. Using one of the sense primers it is possible to obtain a PCR product with Pichia-CYM1-IIIb using high quality genomic DNA as template. This product of ˜2100 bp will be cloned and sequenced. If it fails to produce a band of ˜2100 bp, it would be nessessary to isolate mRNA from Pichia, produce double stranded cDNA and ligate adaptors to the ends as described by the manual to the Clontech Marathon cDNA Amplification Kit (BD (Becton, Dickinson and Company)). Using the two sequence specific sense primers it is possible to obtain the 3′ end of the mRNA of approximately 2600 bp and the sequence specific antisense primers to amplify the 5′ end including the sequence encoding the hypothetical active site, HXXEH motif. Synthesis of sequence specific oligonuleotides from the 5′ and 3′ untranslated region, full-length cDNA encoding the Cym1 orthologue in Pichia can be cloned.

TABLE 3

| | |
|---|---|
| Amino acid sequence | K Y P V R D P |
| Oligo Pichia-CYM1-Ia | 5′AARTAYCCXGTXMGXGAYCC 3′ |
| Amino acid sequence | H P S N A K |
| Oligo Pichia-CYM1-Ib | 3′GTRGGXWSXTTRCGXTTY 5′ |
| Amino acid sequence | D P F F K M |
| Oligo Pichia-CYM1-IIa | 5′GAYCCXTTYTTYAARATG 3′ |
| Amino acid sequence | G V V Y N E M |
| Oligo Pichia-CYM1-IIb | 3′CCXCAXCAXATRTTRCTYTAC 5′ |
| Amino acid sequence | E K G G A Y G |
| Oligo Pichia-CYM1-IIIb | 3′CTYTTYCCXCCXCGXATRCC 5′ |

X-Inosine, Degenerated Oligonucleotides Follow the International Union of Biochemistry (http://www.chem.qmul.ac.uk/iubmb/misc/naseq.html).
Genedisruption of Cym1 Orthologue in Pichia Pastoris or Pichia Methanolica

The sequence encoding the Cym1 orthologue in Pichia should be cloned in a vector like pBluescript-II in HindIII and SacI or a similar vector, if these are not present in the ORF. Insertion of the ORF in HindIII and SacI sites removes most of the multiple cloning sites from the vector, which ease the possibility to find restriction enzyme sites that are only present in the ORF. Cloning of the KanMX casette within the ORF, preferentially so that 1000 bp of the Pichia CYM1 are present on each site of the KanMX cassette creates the Pichia CYM1 disruption cassette. This construct can then be amplified by PCR, using primers at specific for the 5′ and 3′ end of the Pichia CYM1 gene. Transformation of the PCR product into strains of Pichia followed by selection of transformants on YPD plates containing 100 μg/ml geneticin (G-418). Verification of the correct integration into the Pichia genome should be tested by colony PCR, using Pichia sequence specific CYM1 primers that binds 5′ and 3′ to the KanMX cassette. From the size of the PCR product it is possible to distinguish whether the integration event is correct.

Expression of Foreign Proteins and Peptides in Pichia Pastoris

For expression of peptides one could use the expression vector, pPICα (inducible expression) or pGAPZα (constitutive expression) both from Invitrogen. Both these vectors use the preprosequence of the α-mating factor from Saccharomyces cerevisiae to direct the fusion peptide through the secretory pathway. Within the Golgi apparatus the preprosequence of the α-mating factor is removed and the peptide of interest released to the media. If it's proteins that should be expressed, both vectors metioned above can be used without the preprosequence of the α-mating factor (pPICZ and pGAPZ), where the heterologus expressed protein is cytosolic located and can be isolated from intact cells.

Expression of Foreign Proteins and Peptides in Pichia methanolica

Expression of proteins and peptides in Pichia methanolica is performed in a similar manner as in Pichia pastoris, where plasmids are avaiable both for intracellular expression and for secretion to the media. Intracellular expressed proteins can be cloned into pMET (Invitrogen) and for secretion in pMETα (Invitrogen). The pMETα contain the preprosequence of the α-mating factor from Saccharomyces cerevisiae as used for expression in Pichia pastoris. Expression is induced by methanol in this system.

Results

The Influence of Growth Conditions on the CCK-22 Processing

The intra- and extracellular fraction of CCK-22 was measured from BJ2168 expressing proCCK. The intracellular fraction remained unaltered whether the cells were in exponential growth or had reached stationary phase (FIG. 2). However, the relative amount of secreted CCK-22 changed dramatically when the cells reached stationary phase. During exponential growth the fraction of CCK-22 was 23%, but in the stationary phase (after 270 min) the fraction increased to 37% (FIG. 2). Hence, for the experiments described herein only exponentially growing cells were used.

The Significance of the Lys Residue in the Release of CCK-22

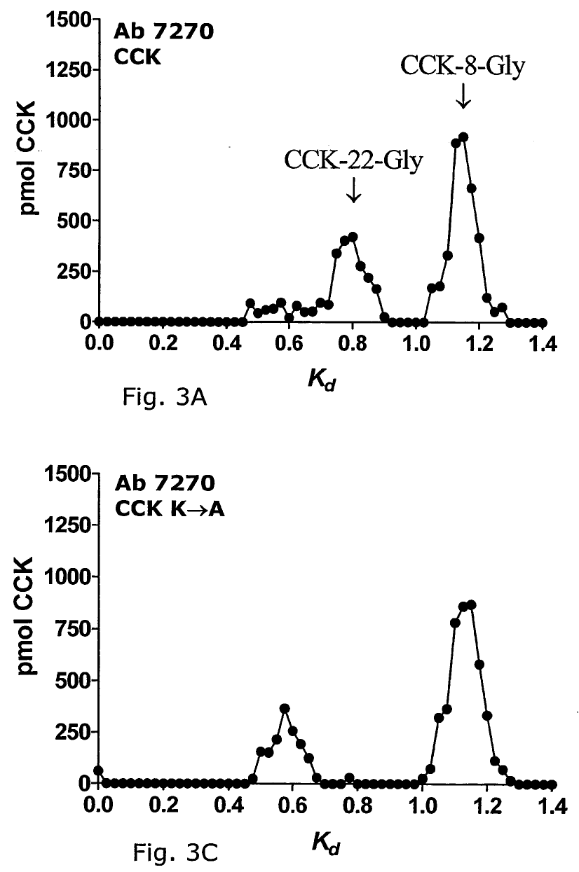

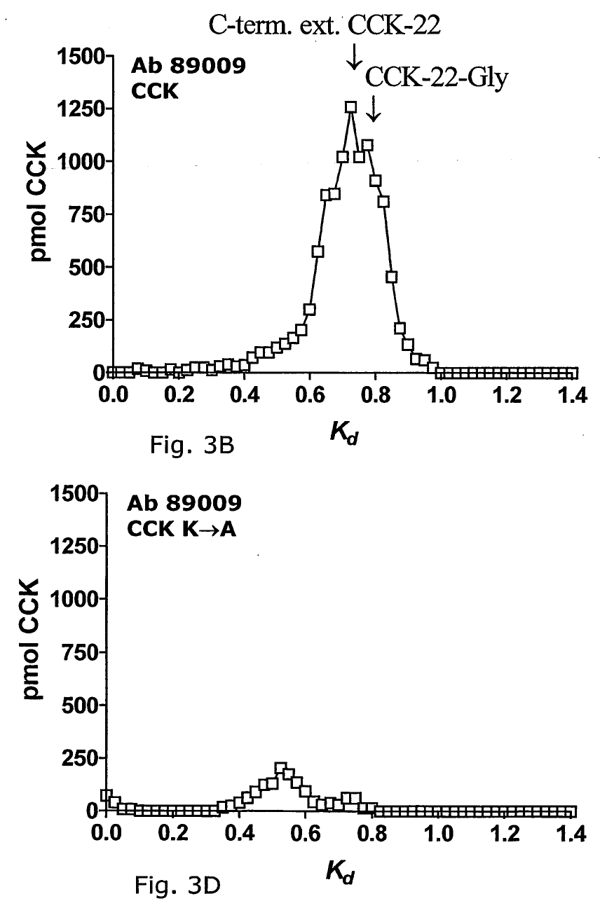

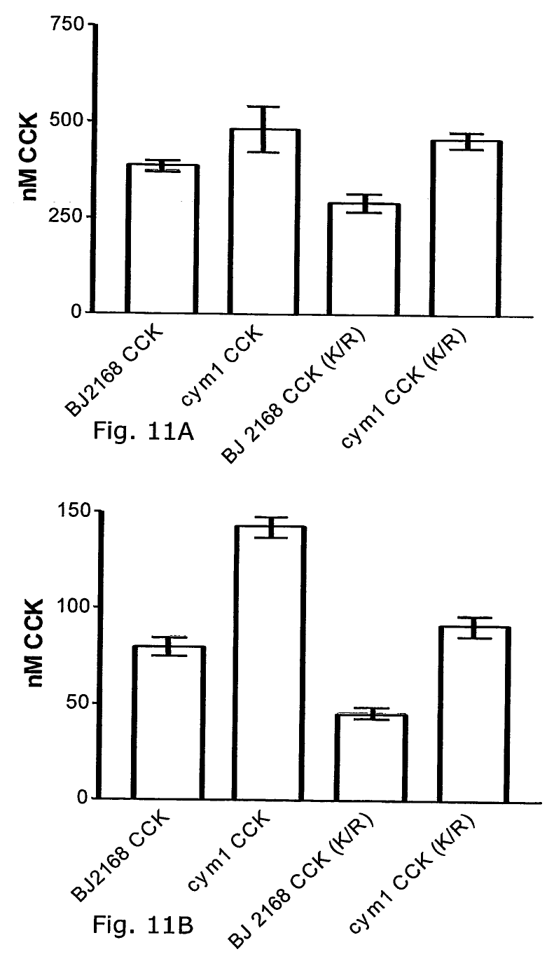

To evaluate the role of the Lys residue in proteolysis, transformants of BJ2168 with the two expression constructs, proCCK and proCCK (K→A) were grown to late exponential phase and the culture media collected. The media from each strain was subjected to gel chromatography and the content of Gly-extended CCK in the collected fractions where measured with Ab 7270. CCK from the wild type media eluted in two major peaks at K_{d}=0.8 and 1.1 (FIG. 3 A) in accordance with the previously established elution positions for CCK-22-Gly and CCK-8-Gly (Cantor et al., 1987; Rourke et al., 1997), while the proCCK (K→A) only gave rise to CCK-8-Gly and a larger form eluting at a K_{d}=0.6 (FIG. 3 C). The two peaks eluting at K_{d}=0.7 and 0.8 for the wt construct (FIG. 3 B) correspond to C-terminally extended CCK-22 and CCK-22-Gly, respectively (Rourke et al., 1997), whereas no CCK-22 immuno-reactivity was observed in these positions for the proCCK (K→A) construct (FIG. 3 D). However, a small peak of immuno-reactivity was seen at K_{d}=0.55 which may be due to the slight cross reactivity of Ab 89009 with a larger unprocessed fragment (Paloheimo et al., 1994). To investigate the effect of substituting Arg for Lys, proCCK (K→R) was transformed into BJ2168. Media from transformants where analysed before and after tryptic cleavage. The fraction of proCCK processed to CCK-22 was similar to that seen for wild type CCK (FIG. 11).

Analysis of Secreted CCK Peptides by Mass Spectrometry

Media collected from BJ2168 transformed with proCCK were analysed by mass spectrometry (FIG. 12). The fragments obtained correspond to the processing leading to CCK-39, CCK-22 and CCK-8. Two peptides were identified N-terminal of Lys⁶, (Tyr⁴⁵-Val⁶⁰, 1805.0 Da and Tyr⁴⁵-Lys⁶¹, 1932.2 Da). It appeared likely that the former was a carboxypeptidase degradation product of the latter. To elucidate this question and in an attempt to identify the C-terminal extended CCKs, the present inventors produced a disruption strain in which both KEX2, encoding the serine protease responsible for the processing to CCK-8, and the carboxypeptidase encoded by KEX1 were mutated. Following transformation of proCCK Into this kex2 kex1 strain (LJY22) and subsequent analysis of the secreted peptides the inventors found only the peak corresponding to Tyr⁴⁵-Lys⁶¹. The same pattern, with only the peak corresponding to Tyr⁴⁵-Lys⁶¹ was seen using single gene disruption of KEX1 and KEX2 to express proCCK (data not shown). Thus the Tyr⁴⁵-Val⁶⁰ must be a degradation product in accordance with CCK-22 arising from cleavage after Lys⁶¹. Additional fragments were discovered by CCK expression in the kex2 kex1 strain corresponding to processing leading to CCK-61 (not identified in mammals), CCK-58, C-terminal extended CCK-39 and C-terminal extended CCK-22 (FIG. 12), whereas none of the peptides corresponding to CCK-8 could be identified, in accordance with our previous work showing that Kex2p is responsible for this processing (Rourke et al., 1997).

Kex2p is Involved in the Biosynthesis of CCK-22

Previous analysis of CCK peptides secreted from a kex2 strain as well as the results obtained by mass spectrometry indicate that the cleavage at Lys⁶¹ releasing CCK-22 can occur without the involvement of Kex2p. However, the kex2 strain shows a decrease in CCK-22 concentration. ProCCK was expressed both in the vacuole protease deficient and the isogenic kex2 strain (BJ2168 and LJY23) and the processed intra- and extracellular fractions of CCK-22 from exponentially growing cells were measured. Approximately 28% of the intracellular CCK content was processed after Lys⁶¹ in BJ2168, whereas only 6% was processed within the kex2 strain. Analysis of secreted CCK peptides showed that the media collected from BJ2168 expressing proCCK contained approximately 20% CCK-22, whereas from the kex2 mutant, the amount was reduced to 5%. These results indicate that Kex2p is involved in the processing leading to CCK-22. However, there are other proteases that can perform the cleavage at LyS61.

In Vitro Assay of Lys⁶¹ Cleavage

To investigate the nature of the protease(s) in addition to Kex2p that are able to perform the endoproteolytic cleavage after the single Lys⁶¹ residue of proCCK, an in vitro assay was established using crude preparations from of S. cerevisiae and synthetic CCK-33 as substrate.

Using extract from the vacuole protease deficient strain, BJ2168, there was an extensive N-terminal degradation, and the recovery of measurable CCK was less than 10% of the control without yeast extract. Because the assay depends on the intact N-terminus of CCK-22 for the antibody to bind, the inventors created a strain where some of the known S. cerevisiae aminopeptidases were deleted. The Y14953 strain (ape1) was used as parental strain in which the APE2 and APE3 genes were also deleted. Using this LJY123 strain to prepare the cell extract there was a 2-3 fold better recovery of immuno-reactivity compared to the recovery seen with BJ2168.

Processing to CCK-22 Depends on Metal Ions

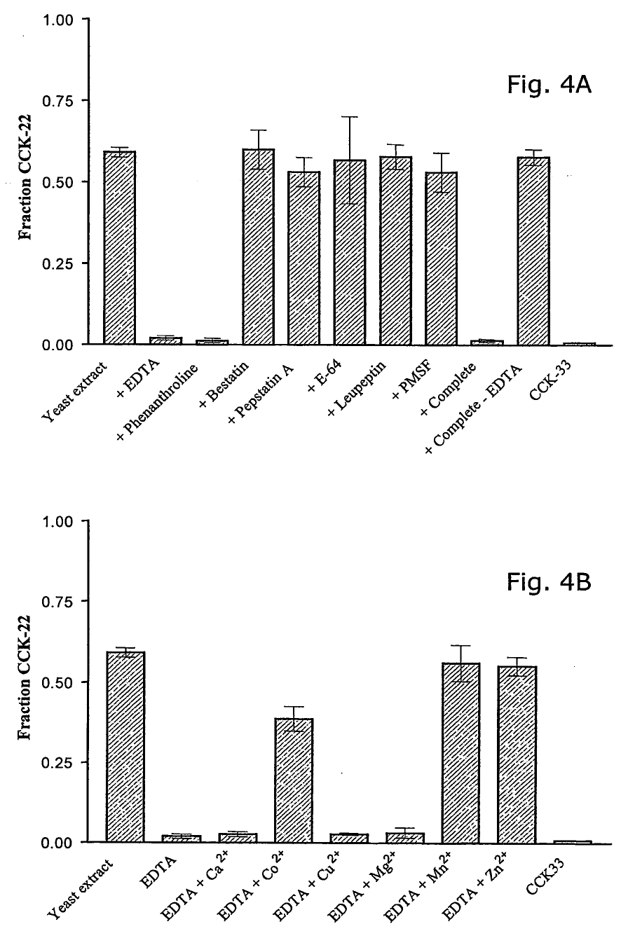

The nature of the protease performing the cleavage of synthetic human CCK-33 to CCK-22 was analysed by inclusion of a number of different inhibitors with the extract from LJY123. The results showed that only the addition of a metal chelating agent inhibited proteolysis of CCK-33 to CCK-22 (FIG. 4 A).

The metal dependency of the protease was tested in vitro, after the activity initially was inhibited by addition of 1 mM EDTA. Reconstitution of the activity leading to maturation of CCK-22 was tested by addition of different divalent cations in 0.2 mM surplus. Addition of Zn²⁺, Co²⁺ and Mn²⁺ could reestablish the protease activity, whereas Ca²⁺, Cu²⁺ or Mg²⁺ had no effect (FIG. 4 B) in accordance with the properties of known metalloproteases, which are only activated by Zn²⁺, Co²⁺ and Mn²⁺. Reactivation using increasing Zn²⁺ concentrations showed a biphasic pattern, with Zn²⁺ acting inhibitory at concentrations above 5 mM (data not shown).

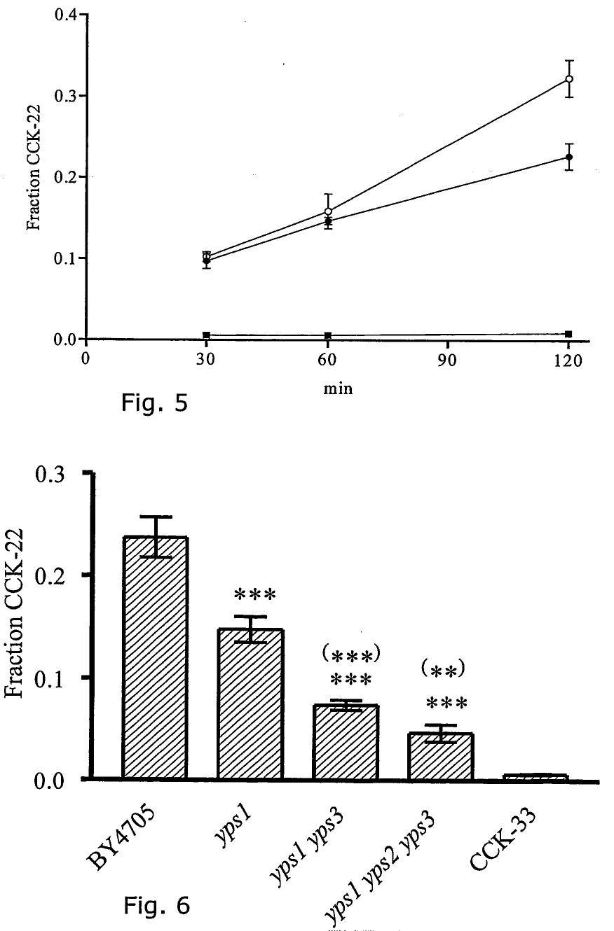

The time course of CCK-cleavage by Zn²⁺ and Mn²⁺ reactivated metalloproteases was analysed using cell extract from LJY123, after initial inhibition with 1 mM EDTA. Reactivation was performed by addition of 1.2 mM Zn²⁺ or Mn²⁺ followed by incubation for 30, 60 and 120 min. In this assay and the following in vitro protease assays the inventors used the N-terminal acetylated CCK-33-Gly (Ac-CCK-33-Gly) as substrate, which resulted in much slower non-specific degradation. Measurement of the CCK-22 immuno-reactivity before and after tryptic cleavage using Ab 89009 showed no difference in the activation potency between Zn²⁺ and Mn²⁺ at 30 and 60 min, however after 120 min 10% more CCK-22 immuno-reactivity was measured using Mn²⁺ as activator compared to Zn²⁺ (FIG. 5). This increase in immuno-reactivity is probably due to an inhibition of degradation following addition of Mn²⁺ here as well as to the yeast cell extracts used in Table 4.

Table 4. Metalloproteases in Saccharomyces cerevisiae. Search performed in Swiss-Prot Sequence Retrieval System (SRS) http://www.expasy.ch/. Protease assay performed in two independent assays (A and B) using extracts from the metalloprotease deficient strains. The amount of CCK-22 is measured with Ab 89009 and the total amount of CCK is measured after tryptic cleavage with Ab 89009. Putative metalloproteases are marked with *.

| | Swiss- | | CCK-22 | | Total CCK | | Fraction | |
|---|---|---|---|---|---|---|---|---|
| | Prot | | [nM] | | [nM] | | CCK-22 | |
| Name | acc # | ORF | A₁ | B₁ | A₂ | B₂ | A₁/A₂ | B₁/B₂ |
| AAP1 | P37898 | YHR047c | 3.2 | 3.2 | 36 | 32 | 0.09 | 0.10 |
| AFG3 | P39925 | YER017c | 2.8 | 2.4 | 23 | 24 | 0.12 | 0.10 |
| APE1 | P14904 | YKL103c | 4.4 | 3.7 | 34 | 28 | 0.13 | 0.13 |
| APE2 | P32454 | YKL157w | | | | | | |
| APE3 | P37302 | YBR286w | | | | | | |
| DPP3 | Q08225 | YOL057w | 4.3 | 4.6 | 31 | 35 | 0.14 | 0.13 |
| LTA4 | Q10740 | YNL045w | 3.9 | 4.0 | 32 | 29 | 0.12 | 0.13 |
| MIP1 | P35999 | YKL134c | 3.4 | 3.3 | 34 | 33 | 0.10 | 0.10 |
| PRD1 | P25375 | YCL057w | 2.4 | 2.8 | 28 | 26 | 0.09 | 0.11 |
| QRI7* | P43122 | YDL104c | 2.8 | 2.9 | 23 | 24 | 0.12 | 0.12 |
| RCA1 | P40341 | YMR089c | 4.2 | 3.5 | 31 | 27 | 0.14 | 0.13 |
| STE23 | Q06010 | YLR389c | 2.6 | 2.3 | 25 | 20 | 0.10 | 0.12 |
| STE24 | P47154 | YJR117w | 3.4 | 2.8 | 19 | 17 | 0.18 | 0.16 |
| YBS4* | P38244 | YBR074w | 2.6 | 2.7 | 27 | 29 | 0.10 | 0.09 |
| YHR3* | P38821 | YHR113w | 2.5 | 2.4 | 26 | 26 | 0.10 | 0.09 |
| YHT2* | P38836 | YHR132c | 3.8 | 3.3 | 34 | 32 | 0.11 | 0.10 |
| YIK8* | P40483 | YIL108w | 5.7 | 5.2 | 43 | 39 | 0.13 | 0.13 |
| YIN7* | P40462 | YIL137c | 3.5 | 2.8 | 23 | 20 | 0.15 | 0.14 |
| YK18* | P36132 | YKR038c | 2.9 | 2.1 | 23 | 20 | 0.13 | 0.11 |
| YME1 | P32795 | YPR024w | 2.3 | 2.7 | 25 | 25 | 0.09 | 0.11 |
| MAS2 | P11914 | YHR024c | ND, Lethal genes | | | | | |
| MAS1 | P10507 | YLR163c | | | | | | |
| AXL1 | P40851 | YPR122w | 3.5 | 3.4 | 31 | 30 | 0.11 | 0.11 |
| CYM1* | P32898 | YDR430c | 0.6 | 0.4 | 42 | 39 | 0.01 | 0.01 |
| YOJ8* | Q12496 | YOL098c | 3.6 | 3.8 | 35 | 34 | 0.10 | 0.12 |

Extracellular Yapsin Activity

To investigate whether any protease activity is secreted or attached extracellularly to the plasma membrane, the protease activity was assayed in media and with intact yeast cells. No degradation of CCK-33 occurred after 1 h of incubation at 30° C. using media from exponential growing LJY123 cells in accordance with earlier observations (Rourke et al., 1997). During incubation with intact yeast cells, cleavage to expose the N-terminus of CCK-22 could be measured (FIG. 6) however, this protease activity could not be abolished by the inhibitors investigated (data not shown). By using intact cells containing gene disruptions of YPS1, YPS2 and YPS3 (Table I) the fraction of processed CCK-22 decreases by deletion of each of the three aspartyl proteases compared to wild type cells (FIG. 6). These data show that the three proteases all have extracellular protease activity, which can cleave at Lys⁶¹ in proCCK. Preliminary results indicate that gene disruption of YPS7 decreases extracellular Lys⁶¹ processing in amounts comparable to the YPS1 deletion (unpublished results).

Expression of proCCK in Metalloprotease Deficient Strains

Based on previously described metalloproteases in S. cerevisiae with endoproteolytic activity (Adames et al., 1995; Schmidt et al., 2000), gene deletion strains of AXL1 (LJY201) and STE24 (LJY202) were initially prepared in BJ2168. ProCCK expression in these strains showed that proteolysis after Lys⁶¹ was unchanged compared to wild type, and it was decided to test the remaining metalloprotease deficient strains LJY123, LJY203, LJY204 and the metalloprotease deficient strains obtained through Euroscarf (Table I) for their ability to secrete CCK-22 (mitochondrial peptidases were not included). The CCK-22 immuno-reactivity did not change significantly among the CCK producing metalloprotease deficient strains (data not shown), and no protease responsible for the processing of heterologous expressed proCCK to CCK-22 was identified by this approach.

CYM1 Encodes a Protease that Can Release the Free N-Terminus of CCK-22

Cell extracts were prepared from each of the viable metalloprotease deficient strains and tested in the in vitro protease assay to investigate whether any reduction in proteolysis was measurable. In this assay 1 mM Mn²⁺ and 1 mM bestatin were included prior to the addition of Ac-CCK-33-Gly, since it was found that the recovery was 80-90% compared to 30% without addition of these aminopeptidase inhibitors (data not shown). Deletion of CYM1 almost abolished the protease activity, whereas none of the other metalloprotease deficient strains showed a significant change in the biosynthesis of CCK-22 (Table 4).

Expression of CYM1 on a Multicopy Plasmid Increases the Fraction of Matured CCK-22 in Vitro

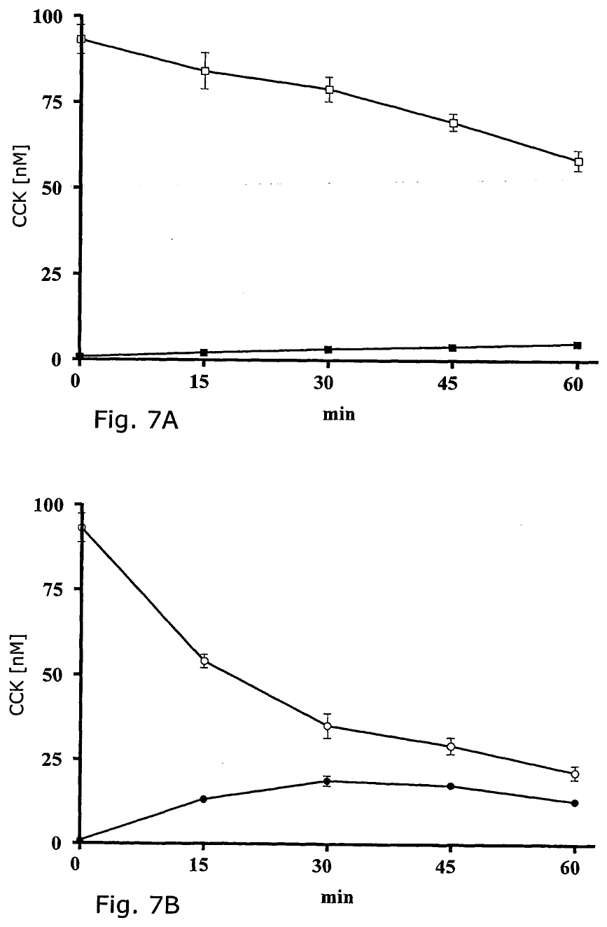

To determine whether the amount of synthetized CCK-22 correlates with the amount of Cym1p in vitro, Cym1p was expressed on a multicopy plasmid and the fraction of synthetized CCK-22 analysed over time. Cell extract from BJ2168 transformed with pRS425 CYM1 and the control transformed with the empty pRS425 vector were used in the in vitro protease assay with 1 mM Mn²⁺ in which the reactions were terminated after 15, 30, 45 and 60 min. The CCK-22 immuno-reactivity was measured with Ab 89009 and the remaining CCK-33 was measured with the same antibody after tryptic cleavage (FIG. 7). Expression of CYM1 on a multicopy plasmid enhanced the rate of CCK-22 production several fold. However, the inventors also observed an increased degradation of CCK-33 and CCK-22 (FIG. 7 B). When the same experiment was performed at pH 6.0 and pH 7.5, there was a dramatically increased degradation and after 30 min incubation the CCK immuno-reactivity was undetectable at pH 6.0 (data not shown). These results show that the Lys-specific cleavage in CCK-22 maturation in vitro is dependent on the amount of Cym1p.

Expression of proCCK in cym1 Mutant Strain Enhances CCK Secretion

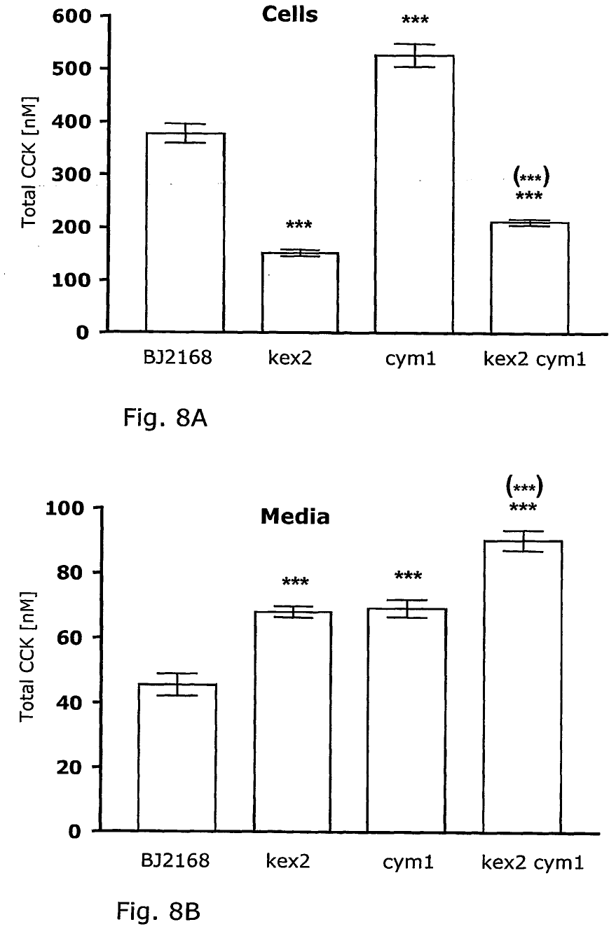

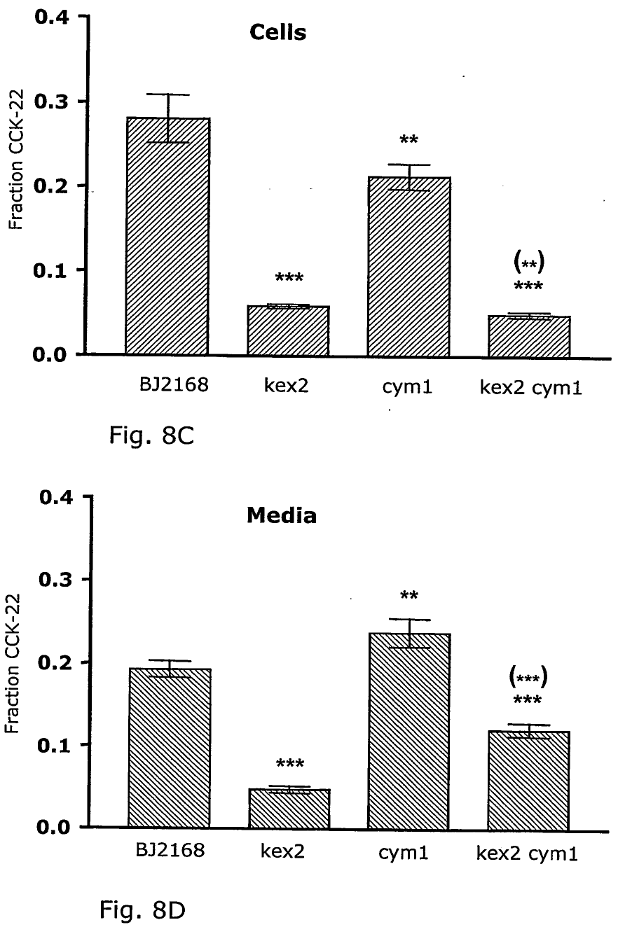

To elucidate the role of CYM1 in the biosynthesis of CCK-22 in vivo, gene deletions of CYM1 were prepared in the vacuole protease deficient strain, BJ2168, and isogenic kex2 strain. Deletion of CYM1 resulted in an approximately 40% increase in the total amount of proCCK within the cells (FIG. 8 A) accompanied by a similar decrease in CCK-22 independent of KEX2 disruption (FIG. 8 C). Also the secreted amount of total CCK in the cym1 strains increased with more than 60% (FIG. 8 B), but unlike the fractional decrease in intracellular CCK-22 there was an increase in the extracellular fractions of CCK-22 compared to vacuole protease deficient strain and the isogenic kex2 strain (FIG. 8 D).

Expression of CCK K→A Mutant Leads to Intracellular CCK Accumulation Comparable to the Accumulation of Wild Type CCK in a cym1 Strain

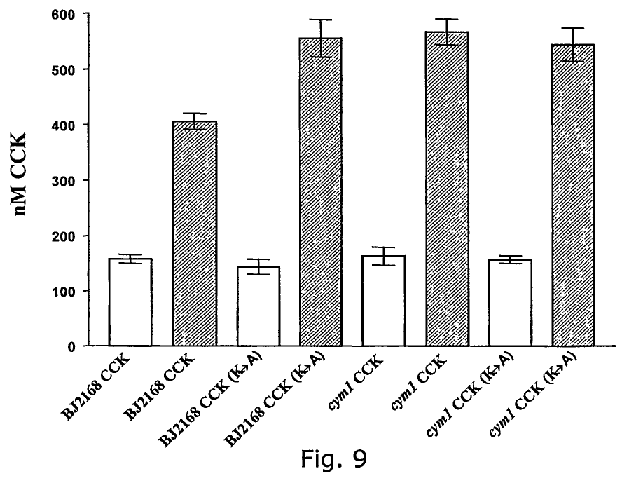

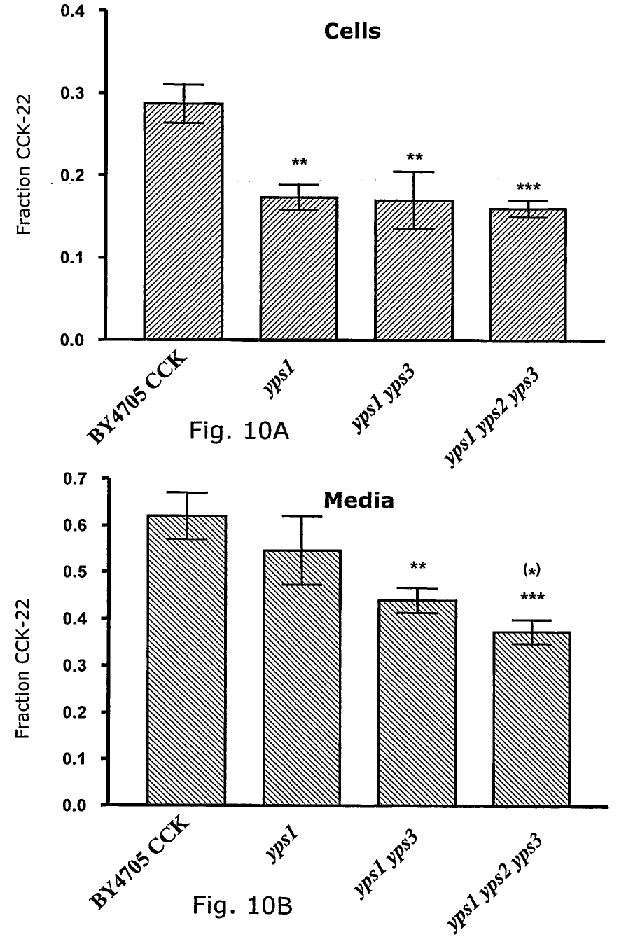

The observations that a gene disruption of CYM1 causes an increase in intracellular concentrations of CCK (FIG. 8 A) raise the question whether the proteolytic activity of Cym1p leads to degradation of CCK-22 prior to translocation into the ER. Therefore, the inventors examined the intracellular CCK content in strains expressing CCK where the maturation of CCK-22 has been eliminated by using the Lys⁶¹→Ala⁶¹ mutant. Transformants of this CCK mutant in the vacuole protease deficient strain, BJ2168 and the isogenic cym1 strain were analysed using Ab 7270 after trypsin and carboxypeptidase B treatment and there was an increase in the intracellular CCK immuno-reactivity for this construct compared to expression of wild type CCK (FIG. 9). Mutant CCK (K→A) and wild type CCK transformants resulted in an increase in the intracellular proCCK concentration when expressed in BJ2168 and the CYM1 disruption strain, respectively. The increase in intracellular proCCK was not additive showing that proteolytic activity of Cym1p leads to degradation of CCK-22 prior to translocation into the ER.

Expression of proCCK in Aspartyl Protease Deficient Strains

The Lys⁶¹-specific cleavage of proCCK was analysed in null mutants of YPS1, -2 and -3, where the intra- and extracellular amount of CCK-22 was measured from exponentially growing cells of wild type yeast, BY4705 and the isogenic aspartyl protease deficient strains transformed LJY13, -14 and -15 with proCCK. Both intra- and extracellular CCK immuno-reactivity of BY4705 was lowered more than 10 fold compared to the vacuolar protease deficient strain, BJ2168 (data not shown). The intracellular fraction of CCK-22 decreased significantly from approximately 28% in wild type cells to 17% In the yps1 strain, whereas no additional decrease could be measured by gene disruptions of YPS2 and YPS3 (FIG. 10 A). The extracellular fraction of CCK-22 did however show that Yps1p, Yps2p and Yps3p all are involved in the biosynthesis of CCK-22 and that the triple mutant reduced the fraction of CCK-22 to ⅔ compared to wild type yeast (FIG. 10 B).

CYM1 Disruption Leads to a Two-Fold Increase in the Total Amount of Secreted Wild Type CCK as Well as the CCK K→R Mutant

To elucidate whether Cym1p cleaves C-terminally to a single Arg residue, the CCK (K→R) mutant was expressed in the vacuolar protease deficient strain, BJ2168 and the isogenic cym1 strain. The concentration of both intra- and extracellular CCK was compared to wild type CCK expressed in these strains. The total amount of the mutated CCK (K→R) was increased both intra- and extracellular comparable to wild type CCK (FIG. 11). Both wild type CCK and the Lys⁶¹→Arg⁶¹ mutant showed more than a two-fold increase in the measurable amount of extracellular CCK when expressed in the cym1 strain (FIG. 11 B).

Usage of Cym1p Activity in the Synthesis of Peptides

Another aspect of the invention is to use the activity from Cym1p, either expressed from its own promoter or from a strong constitutive promoter such as PGK1, ADH1 or TPI1, or the induceable GALL promoter to produce an increased amount cytosolic Cym1p activity. As previously mentioned, the synthesis of CCK-22 is significantly increased when CYM1 is transcribed from its own promoter on a 2μ plasmid (FIG. 7). Transcription can either be performed from a plasmid containing the promoter, CYM1 and a terminator, or by introducing the desired promoter into the genome by heterologous recombination to substitute the endogenous promoter of CYM1.

The activity can be used intracellularly to generate peptides that do not require post-translational modifications from the secretory pathway, such as disulfide bond formation, N- and O-glycosylation or exoprotease activity.

The role of Cym1p cytosolic activity in intracellular peptide synthesis is shown in the biosynthesis of CCK-22 in wild type cells compared to the isogenic strain with a CYM1 disruption, which shows a significant increase in the amount of CCK-22 (FIG. 8C). Synthesis of the peptide of interest should be performed in such a way that translocation into the endoplasmatic reticulum (ER) is avoided. This can be performed either by removal of the hydrophobic amino-terminal signal sequence from proteins that enter the ER post-translationally, or by expression in a temperature sensitive secretory mutant such as sec61, which abolishes translocation of secretory peptides into the ER when the temperature is elevated to 37° C.

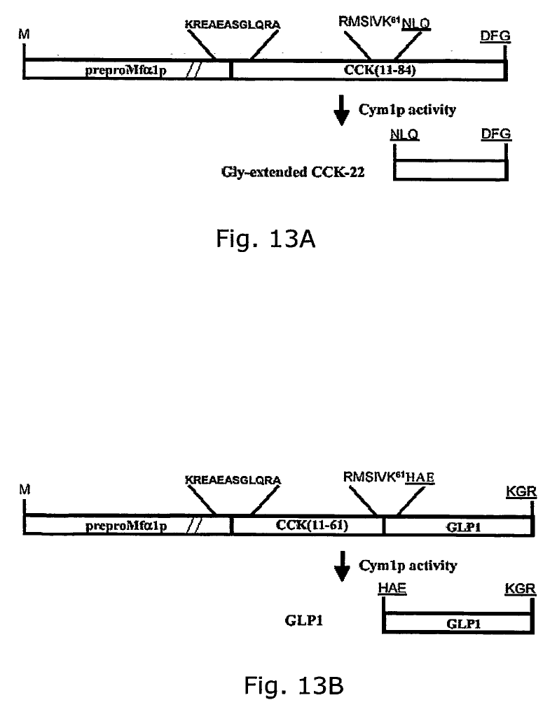

The propeptide or prepropeptide of interest will then be cytosolicly located and a potential substrate for Cym1p. Release of the peptide from its precursor will be carried out by the Cym1p activity by introduction of the cleavage site seen from proCCK, which results in the release of Gly-extended CCK-22 after endoproteolytic processing C-terminal to Lys⁶¹ (Ser-Ile-Val-Lys⁶¹⇓) (FIG. 13A). If the peptide of interest is GLP1, synthesis can be performed as a fusion to a Cym1p cleavage site, which could be part of proCCK (FIG. 13B). The peptide of interest will then accumulate in the cytosol and can be purified from sedimented cells after lysis.

Expression of proBNP in cym1 Mutant Strain Enhance proBNP Secretion

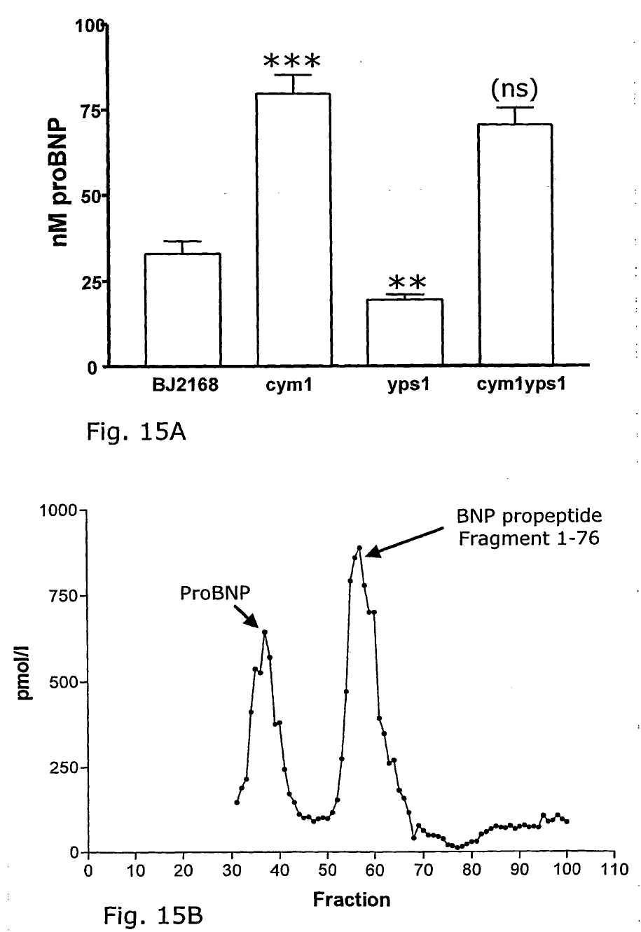

To elucidate the role of CYM1 in the biosynthesis of proBNP in vivo, proBNP was expressed in the the vacuole deficient strain, BJ2168 and the three protease deficient isogenic strains, Δcym1::LEU2 (LJY430), Δyps1::TRP1 (LJY440) and a Δcym1::LEU2 Δyps1::TRP1 (LJY431). Deletion of CYM1 resulted in an approximately 100% increase in the total amount of secreted proBNP, whereas the proBNP secretion was independent on disruption of the gene encoding the aspartyl protease, Yps1p and was thereby similar to the wildtype strain (FIG. 15A). Disruption of both Cym1 and Yps1 was as expected similar to the secreted amount in a cym1 mutant (FIG. 15A).

Analysis of the proBNP in Secreted from a cym1 Mutant

To analyse the proBNP expressed in Saccharomyces cerevisiae, media from a cym1 mutant was applied to FPLC chromatography and analysed by RIA using Ab. 98192. The peak eluting from fraction 34-39 corresponds to intact proBNP, whereas the peak eluting in fraction 53-62 is a processed form of proBNP, most likely the proBNP fragment 1-76 (FIG. 15B). The release of fragment 1-76 and BNP-32 from proBNP, is due to cleavage after a single Arg residue and is probably due to either Kex2 or Yps1 activity.

Discussion

The secreted polypeptides varies with the growth conditions, the fraction of CCK-22 increasing when the culture reaches stationary phase, while the intracellularly processed fraction remains unaltered under stress conditions. The increase in extracellular cleavage to CCK-22 as the cells enter stationary phase could indicate that extracellular endoproteases with the ability to process proCCK to CCK-22 are secreted or expressed on the cell membrane. It is known that the aspartyl proteases, Yps1p and Yps2p, exhibit cell surface activity (Komano et al., 1998). In addition, it has previously been shown that heterologous peptide expression in a yps1 strain improved the recovery of proteins and peptides like albumin, glucagon, GLP1, GLP2 and CART by inhibiting proteolysis C-terminal to mono-basic residues (Egel-Mitani et al., 2000; Kerry-Williams et al., 1998). Thus, recent studies ((Egel-Mitani et al., 2000; Kerry-Williams et al., 1998) and those of the present inventors) show the importance of collecting secreted peptides during exponential growth in order to avoid additional extracellular processing.

ProCCK expressed in a vacuole protease deficient strain showed 30% intracellular processing at Lys⁶¹ in proCCK. The fraction of extracellular Lys⁶¹-processing is, however, decreased to ⅔ of the observed fraction within intact yeast cells, which reveals an intracellular degradation of CCK-22 prior to secretion. The increase in extracellular proteolysis under limited nutrient resources is probably due to an activation or upregulation in transcription of the extracellular proteases under limited nutrient resources as seen with the upregulatlon of YPS1 transcription during stationary phase (Gasch et al., 2000). Part of the cell surface activity can be assigned to the yapsins, Yps1p, Yps2p and Yps3p, but some extracellular activity was sustained even in the triple mutant.

In the present study, the inventors have shown that deletion of KEX2 causes a 5 fold reduction in both the intracellular and extracellular Lys⁶¹-cleavage. The kex2 strain expressing proCCK do not only alter the cleavage of Lys⁶¹ in proCCK, it also changes the intracellular retention time of CCK as the intracellular concentration of CCK peptides is reduced with more than 60%, while the extracellular CCK concentration is increased by almost 60% compared to wild type yeast. Moreover, analysis of the secreted CCK peptides from the kex1 kex2 double mutant and the kex2 mutant showed disappearance of the Tyr⁴⁵-Val⁶⁰ degradation product. Thus, the removal of Lys⁶¹ by Kex1p was abolished in a kex2 strain indicating an enhanced secretion rate through the trans-Golgi network. These results and the observations on the rapid secretion of proCCK suggest that it may be the intracellular retention caused by Kex2p that leads to an increased synthesis of CCK-22 in wild type yeast by Yps1p and probably to some extent by Kex2p.

The type of protease responsible for the intracellular maturation of CCK-22 was investigated in an in vitro protease assay using a crude extract of S. cerevisiae to analyse the processing of synthetic human CCK-33 to CCK-22 in the presence of different inhibitors. By not including detergents in extraction of protease activity, activity from Kex2p as well as the GPI-anchored yapsins was avoided (Azaryan et al., 1993; Fuller et al., 1989; Komano et al., 1999). Of the inhibitors tested, the proteolysis was only inhibited by EDTA and 1,10 ortho-phenanthroline, and the activity could be restored by addition of the divalent cations Zn²⁺, Co²⁺ and Mn²⁺. This indicated that a metalloprotease participates in the maturation of CCK-22.

None of the candidate metalloproteases contain an obvious signal peptide to direct the protein into ER. Therefore, the inventors investigated strains deficient in each of the metalloproteases with the exception of mitochondrial proteases. Expression of proCCK in each of the strains resulted in unaltered maturation of CCK-22 similar to that seen in wild type yeast. However, by using the in vitro protease assay the inventors identified Cym1p as an endoprotease performing post-Lys cleavage of CCK-33. That Cym1p can cleave Lys⁶¹ in proCCK was verified by overexpression studies, showing a several fold increase in enzyme activity.

Intracellular synthesis of CCK-22 was decreased in a cym1 strain accompanied by an increased concentration of total proCCK. In contrast, the fraction of extracellular CCK-22 was increased compared to wild type yeast with a parallel increase in total CCK. These findings are in accordance with a cytosolic location of the Cym1p activity like most insulin-degrading enzymes (Bal et al., 1996) and show that it acts on the preproMfα1p-proCCK construct prior to translocation into the endoplasmatic reticulum. Thus, the pre-translocational degradation of proCCK is decreased by CYM1 disruption and the total production increased.

Expression of proBNP as a fusionpeptide to the preproMfα1p sequence in a cym1Δ mutant shows a two-fold increase of the extracellular proBNP content compared to the wild type strain. Analysis of the secreted proBNP by chromatography disclosed that two major forms were present. One is the entire proBNP, whereas the other is the proBNP fragment 1-76, thus the biologically active BNP-32 is also synthesised, though not detectable in the present assay. The release of proBNP fragment 1-76, most likely depends on the Kex2p activity, however this could not be tested in the present assay, since the release of proBNP depends on both Kex2p and Kex1p.

All publications discussed above are incorporated herein in their entirety.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Adames, N., Blundell, K., Ashby, M. N., and Boone, C. (1995). Science 270:464-67.
Azaryan, A. V., Wong, M., Friedman, T. C., Cawley, N. X., Estivariz, F. E., Chen, H. C., and Loh, Y. P. (1993). J. Biol. Chem. 268:11968-75.
Bai, J. P., Hong, H. J., Rothenberger, D. A., Wong, W. D., and Buls, J. G. (1996). J. Pharm. Pharmacol. 48:1180-84.
Becker, A. B and Roth, R. A. (1992) PNAS 89: 3835-3839.
Brachmann, C. B., Davies, A., Cost, G. J., Caputo, E., Li, J., Hieter, P., and Boeke, J. D. (1998). Yeast 14:115-132.
Cantor, P. and Rehfeld, J. F. (1987). Clin. Chim. Acta 168:153-58.
Egel-Mitani, M., Andersen, A. S., Diers, I., Hach, M., Thim, L., Hastrup, S., and Vad, K. (2000). Enzyme Microb. Technol. 26:671-77.
Egel-Mitani, M., Flygenring, H. P., and Hansen, M. T. (1990). Yeast 6:127-137.
Elbashir, S. M., Harborth, J., Lendeckel, W., Yalcin, A., Weber, K., and Tuschl, T. (2001). Nature 411, 494-98.
Fuller, R. S., Brake, A., and Thorner, J. (1989). Proc. Natl. Acad. Sci. USA. 86:1434-38.
Gasch, A. P., Spellman, P. T., Kao, C. M., Carmel-Harel, O., Eisen, M. B., Storz, G., Botstein, D., and Brown, P. O. (2000). Mol. Biol. Cell 11:4241-57.
Gietz, R. D., Schiestl, R. H., Willems, A. R., and Woods, R. A. (1995). Yeast 11:355-60.
Gøtze J P, Kastrup J, Pedersen F and Rehfeld J F. (2002) Quantification of pro-B-type natriuretic peptide and its products in human plasma by use of an analysis independent of precursor processing. Clin Chem. July; 48(7):1035-42).
Haseloff, J. and Gerlach, W. L. (1988). Nature, 334: 585-91.
Hilsted, L. and Rehfeld, J. F. (1986). Anal. Blochem. 152:119-26.
Hooper N. M. (1994) FEBS Letters 354: 1-6.
Horton, R. M., Ho, S. N., Pullen, J. K., Hunt, H. D., Cai, Z., and Pease, L. R. (1993). Methods Enzymol. 217:270-79.
Jones, E. W. (1991). Methods Enzymol. 194:428-53.
Julius, D., Schekman, R., and Thorner, J. (1984). Cell 36:309-18.
Kerry-Williams, S. M., Gilbert, S. C., Evans, L. R., and Balance, D. J. (1998). Yeast 14:161-69.
Komano, H., Rockwell, N., Wang, G. T., Krafft, G. A., and Fuller, R. S. (1999). J. Biol. Chem. 274:24431-37.
Komano, H., Seeger, M., Gandy, S., Wang, G. T., Krafft, G. A., and Fuller, R. S. (1998). J. Biol. Chem. 273:31648-51.
Needleman, S. B. and Wunsch, C. D. (1970). J. Mol. Biol. 48: 443-53.
Paloheimo, L. I. and Rehfeld, J. F. (1994). Clin. Chim. Acta 229:49-65.
Philippsen, P., Stotz, A., and Scherf, C. (1991). Methods Enzymol. 194:169-82.
Rawlings, N. D. and Barrett, A. J. (1995). Meth. Enzymol. 248:183-228.
Rothstein, R. (1991). Methods Enzymol. 194:281-301.
Rourke, I. J., Johnsen, A. H., Din, N., Petersen, J. G. L., and Rehfeld, J. F. (1997). J. Biol. Chem. 272:9720-27.
Sambrook, J., Fritsch, E. F., and Maniatis, T. (1989). Molecular Cloning: A Laboratory Manual, 2nd Ed., Cold Spring Harbor Laboratory, Cold Spring Harbor, N.Y.
Schmidt, W. K., Tam, A., and Michaelis, S. (2000). J. Biol. Chem. 275:6227-33.
Shippy, R., Lockner, R., Farnsworth, M. and Hampel, A. (1999). Mol. Biotech. 12: 117-29.
Waterhouse, P. M., Graham, M. W., and Wang, M.-B. (1998). Proc Natl Acad Sci USA, 95: 13959-64.

1. A method for producing a protein of interest in a host cell, wherein said host cell has been genetically modified in order to express significantly reduced levels of a metalloprotease comprising a HXXEH motif (SEQ ID NO 1), compared to the corresponding non-modified cell when cultured under identical conditions, the method comprising
a) introducing into the host cell a nucleic acid sequence encoding the protein of interest,
b) cultivating the host cell of step (a) in a suitable growth medium for production of the protein of interest, and
c) isolating the protein of interest.

2. A method according to claim 1, wherein the metalloprotease further comprises a glutamic acid residue between 70 and 80 amino acids C-terminal of the second His residue in the HXXEH motif.

3. A method according to claim 1, wherein the metalloprotease further comprises a glysine residue 3 amino acids N-terminal of the first His residue in the HXXEH motif.

4. A method according to claim 1, wherein the metalloprotease further comprises a glysine residue 5 amino acids C-terminal of the second His residue in the HXXEH motif.

5. A method according to claim 1, wherein the metalloprotease further comprises a lysine residue 8 amino acids C-terminal of the second His residue in the HXXEH motif.

6. A method according to claim 1, wherein the metalloprotease further comprises a tyrosine residue 9 amino acids C-terminal of the second His residue in the HXXEH motif.

7. A method according to claim 1, wherein the metalloprotease further comprises a proline residue 10 amino acids C-terminal of the second His residue in the HXXEH motif.

8. A method according to claim 1, wherein the metalloprotease further comprises the consensus sequence SEQ ID NO 2.

9. A method according to claim 1 wherein the metalloprotease further comprises the consensus sequence SEQ ID NO 3.

10. A method according to claim 1, wherein the metalloprotease further comprises a NAXTXXXXT motif between 20 and 30 amino acids C-terminal of the second His residue in the HXXEH motif.

11. A method according claim 1, wherein the metalloprotease is selected from:
i) any one of the group consisting of SEQ ID NO's 4 to 15, and
ii) a sequence which is at least 80% identical to any one of SEQ ID NO's 4 to 15.

12. A method according to claim 1, wherein the metalloprotease is at least 80% identical to the SEQ ID NO: 4.

13. A method according to claim 1, wherein the total amount of the protein of interest is increased at least 5% compared the corresponding non-modified cell when cultured under identical conditions.

14. A method according to claim 1, wherein the total amount of the protein of interest is increased at least 50% more than the corresponding non-modified cell when cultured under identical conditions.

15. The method according to claim 1, in which the host cell is a prokaryotic cell.

16. The method according to claim 1, in which the host cell is a eukaryotic cell.

17. The method according to claim 16, in which the host cell is a non-filamentous fungal cell.

18. The method according to claim 16, in which the host cell is a filamentous fungal cell.

19. The method according to claim 17, in which the host cell is a strain of Saccharomycces.

20. The method according to claim 19, in which the host cell is Saccharomyces cerevisiae.

21. A host cell useful for the expression of a protein of interest, wherein said cell has been genetically modified in order to express significantly reduced levels of a metalloprotease comprising a HXXEH motif (SEQ ID NO 1) than the corresponding non-modified cell when cultured under identical conditions.

22. A host cell according to claim 21, wherein the metalloprotease further comprises the consensus sequence SEQ ID NO 3.
